(19) United States
(12) Patent Application Publication
(10) Pub. No.: US 2013/0261660 A1
(43) Pub. Date: Oct. 3, 2013

(54) MEDICAL DEVICES AND METHODS FOR INSERTING AN ADHESIVE MEMBRANE

(71) Applicant: William F. McKay, Memphis, TN (US)

(72) Inventor: William F. McKay, Memphis, TN (US)

(73) Assignee: Warsaw Orthopedic, Inc., Warsaw, IN (US)

(21) Appl. No.: 13/438,637

(22) Filed: Apr. 3, 2012

(51) Int. Cl. A61B 17/03

(52) U.S. Cl. 606/213

(57) ABSTRACT

Improved medical devices and methods are provided that deliver an adhesive sheet or membrane at or near a target tissue site, the medical devices and methods comprise a cannula defining a longitudinal axis and having a proximal end and a distal end, the proximal end of the cannula having an opening configured to receive a flowable material, the distal end of the cannula connected to an expandable member, the expandable member having an interior configured to receive the flowable material and a surface aligned with and contacting at least a portion of the adhesive membrane, the adhesive membrane configured to attach to a select tissue surface of the target tissue site in an open position.

### BACKGROUND

Intervertebral discs lie between adjacent vertebrae of a spine. An intervertebral disc includes fibrosus bands, which provide cushion to facilitate motion of the vertebrae and spacing of the vertebrae from nerves and vessels. The fibrosus bands include an outer annulus fibrosus, which is the peripheral portion of an intervertebral disc and defines an inner nucleus pulposus. The nucleus pulposus includes loose fibers suspended in a gel substance having a jelly like consistency that absorbs impacts to the body while keeping the vertebrae separated.

Intervertebral discs may be displaced or damaged due to disease or aging. Disruption of the annulus fibrosus can allow the nucleus pulposus to protrude into the vertebral canal or intervertebral foramen, a condition known as a herniated or slipped disc. A rupture in the annulus fibrosis can allow the escape of nucleus pulposus components. The extruded nucleus pulposus may press on a spinal nerve, which may result in nerve damage, pain, numbness, muscle weakness and paralysis. Furthermore, as a disc dehydrates and hardens due to age or disease, the disc space height will be reduced, leading to instability of the spine, decreased mobility and pain. Moreover, excessive movement of the spinal segments caused by the disc space height reduction could weaken the annulus fibrosus and in certain cases, tear it.

Non-surgical treatments, such as medication, rehabilitation and exercise can be effective, however, sometimes these treatments may fail to relieve the symptoms associated with the above conditions.

Surgical treatment of spinal disorders can include discectomy, laminectomy, fusion and implantable prosthetics. These surgical treatments involve penetrating the annulus fibrosus with surgical instruments and often implanting medical devices at or near the site of injury. Other treatments require forcing apart vertebrae with a balloon so that a thermoplastic material can be injected into the disc space. These treatments, however, may suffer from disadvantages and drawbacks. For example, the treatments may cause damage to the annulus fibrosus and cause the nucleus pulposus to leak out from the annulus fibrosis causing more nerve damage, pain, numbness, muscle weakness and, in severe cases, paralysis.

Therefore, it would be desirable to provide medical devices and methods that include a seal that effectively forms a seal with the surrounding disc tissue to prevent or minimize leakage of the nucleus pulposus during treatment. Desirably, the medical devices and methods prevent or minimize leakage through the annulus fibrosus of the intervertebral disc.

### SUMMARY

New medical devices and methods are provided that allow insertion of membranes by the surgeon to easily seal the target tissue site and improve healing in a minimally invasive procedure. In some embodiments, the medical devices and methods comprise an expandable member that when partially or completely inflated applies pressure to the membrane and allows it to conform to and seal the target tissue site to improve healing. In some embodiments, the membrane can comprise a sheet containing an adhesive to enhance binding to and sealing the target tissue site (e.g., annulus fibrosus of the intervertebral disc). In some embodiments, the membrane can comprise a therapeutic agent to further enhance healing of the target tissue site and/or reduce pain.

In some embodiments, a medical device for delivering an adhesive membrane adjacent to a target tissue site is provided, the medical device comprising a cannula defining a longitudinal axis and having a proximal end and a distal end, the proximal end of the cannula having an opening configured to receive a flowable material, the distal end of the cannula connected to an expandable member, the expandable member having an interior configured to receive the flowable material and a surface aligned with and contacting at least a portion of the adhesive membrane, the adhesive membrane configured to attach to a select tissue surface of the target tissue site in an open position.

In some embodiments, a medical device for delivering an adhesive sheet adjacent to a target tissue site is provided, the medical device comprising a cannula defining a longitudinal axis and having a proximal end and a distal end, the proximal end of the cannula having an opening configured to receive a flowable material, the distal end of the cannula connected to a balloon, the balloon having an interior configured to receive the flowable material and a surface aligned with and contacting at least a portion of the adhesive sheet, the balloon configured to move in an inflated position and an a deflated position, wherein in the inflated position, the adhesive sheet attaches to a select tissue surface of the target tissue site and in the deflated position, the adhesive sheet is in a closed position.

In some embodiments, a method for treating a target tissue site is provided, the method comprising: inserting a balloon adjacent to the target tissue site, the balloon having a surface aligned with and connected to at least a portion of an adhesive sheet, the balloon configured to move from a deflated position when the adhesive sheet is in a closed position to an inflated position when the adhesive sheet is in an open position; positioning the adhesive sheet adjacent to a select tissue surface of the target tissue site; inflating the balloon to move the adhesive sheet to an open position so as to attach the adhesive sheet to the select tissue surface and adhere the adhesive sheet to the select tissue surface of the target tissue site.

The medical device may: (i) consist of one or more adhesives and one or more therapeutic agents (or one or more of its pharmaceutically acceptable salts, esterified forms or non-esterified forms thereof), and one or more biodegradable polymer(s); or (ii) consist essentially of one or more adhesives and one or more therapeutic agents (or one or more of its pharmaceutically acceptable salts, esterified forms or non-esterified forms thereof), and one or more biodegradable polymer(s); or (iii) comprise one or more adhesives and one or more therapeutic agents (or one or more of its pharmaceutically acceptable salts, esterified forms or non-esterified forms thereof), and one or more biodegradable polymer(s); or (iv) consist essentially of one or more adhesives and one or more therapeutic agents (or one or more of its pharmaceutically acceptable salts, esterified forms or non-esterified forms thereof), and one or more biodegradable polymer(s), and one or more other active ingredients, surfactants, excipients or other ingredients or combinations thereof. When there are other active ingredients (e.g., surfactants, pore forming agents, plasticizers, lubricants, excipients or other ingredients or combinations thereof) in the membrane, in some embodiments these other compounds or combinations thereof comprise less than 50 wt. %. less than 40 wt. %, less than 30 wt. %, less than 20 wt. %, less than 19 wt. %, less than 18 wt. %, less than 17 wt. %, less than 16 wt. %, less than 15 wt. %, less than 14 wt. %, less than 13 wt. %, less than 12 wt. %, less than 11 wt. %, less than 10 wt. %, less than 9 wt. %, less than 8 wt. %, less than 7 wt. %, less than 6 wt. %, less than 5 wt. %, less than 4 wt. %, less than 3 wt. %, less than 2 wt. %, less than 1 wt. % or less than 0.5 wt. %.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWING

In part, other aspects, features, benefits and advantages of the embodiments will be apparent with regard to the following description, appended claims and accompanying drawing where:

FIG. 1 illustrates a side view of one embodiment of the medical device comprising a cannula and an expandable member (e.g., balloon) at its distal end. The distal end of the cannula contains an expandable member that allows insertion of the membrane. The membrane is configured to be attached to a target tissue site. The expandable member is shown in its deflated position and the adhesive sheet is shown in its closed position.

FIG. 2 illustrates a side view of one embodiment of the medical device containing an expandable member that when in an inflated position opens the adhesive sheet. In this way, on pressure to the adhesive sheet, it can attach to and seal a target tissue site.

FIG. 3 illustrates a top view of one embodiment of the cannula that can receive fluid material to inflate the expandable member.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “1 to 10” includes any and all subranges between (and including) the minimum value of 1 and the maximum value of 10, that is, any and all subranges having a minimum value of equal to or greater than 1 and a maximum value of equal to or less than 10, e.g., 5.5 to 10.

### DEFINITIONS

It is noted that, as used in this specification and the appended claims, the singular forms “a,” “an,” and “the,” include plural referents unless expressly and unequivocally limited to one referent. Thus, for example, reference to “a medical device” includes one, two, three or more medical devices.

The term “implantable” as utilized herein refers to a biocompatible medical device (e.g., adhesive sheet or membrane) retaining potential for successful placement within a mammal. The expression “implantable medical device” or “implantable adhesive sheet” “implantable adhesive membrane” and expressions of the like import as utilized herein refers to an object implantable through surgery, injection, or other suitable means whose primary function is achieved either through its physical presence or mechanical properties.

The term “sheet” or “membrane” includes a three-dimensional article with a thickness that is considerably less than its other dimensions. Such an article may alternatively be described as a patch, film, strip or ribbon. In some embodiments, the sheet has an overall thickness of from 0.01 to 1 mm. In some embodiments, the sheet has an overall thickness of from 0.015 to 0.05 mm. In some embodiments, the sheet can be rolled or flat. In some embodiments, the sheet or membrane may comprise a therapeutic agent disposed on one surface or opposed surfaces of it. The therapeutic agent may be uniformly distributed throughout the sheet or membrane or may be disposed in one or more layers.

The term “drug” as used herein is generally meant to refer to any substance that alters the physiology of a patient. The term “drug” may be used interchangeably herein with the terms “therapeutic agent,” “therapeutically effective amount,” and “active pharmaceutical ingredient”, “API”, or “biological agent.” It will be understood that unless otherwise specified a “drug” formulation may include more than one therapeutic agent, wherein exemplary combinations of therapeutic agents include a combination of two or more drugs. The drug provides a concentration gradient of the therapeutic agent for delivery to the site. In various embodiments, the medical device provides an optimal drug concentration gradient of the therapeutic agent at a distance of up to about 0.01 cm to about 20 cm from the administration site. A “drug depot” includes but is not limited to capsules, coatings, matrices, wafers, sheets, strips, ribbons, pills, pellets, microspheres, nanospheres, or other pharmaceutical delivery or a combination thereof. Suitable materials for the depot are ideally pharmaceutically acceptable biodegradable and/or any bioabsorbable materials that are preferably FDA approved or GRAS materials. These materials can be polymeric or non-polymeric, as well as synthetic or naturally occurring, or a combination thereof. Typically, the sheet, membrane and/or depot will be a solid or semi-solid formulation comprised of a biocompatible material that can be biodegradable.

A “therapeutically effective amount” or “effective amount” is such that when administered, the drug results in alteration of the biological activity, such as, for example, inhibition of inflammation, inhibition of pain, and/or improvement in the healing wound, etc. The dosage administered to a patient can be as single or multiple doses depending upon a variety of factors, including the drug's administered pharmacokinetic properties, the route of administration, patient conditions and characteristics (sex, age, body weight, health, size, etc.), extent of symptoms, concurrent treatments, frequency of treatment and the effect desired. In some embodiments, all or parts (e.g., surfaces, regions, layers, etc.) of the medical device (e.g., membrane, sheet) may be designed for immediate release. In other embodiments the medical device (e.g., membrane, sheet) may be designed for sustained release. In other embodiments, the medical device e.g., membrane, sheet) comprises one or more immediate release surfaces, layers, regions and one or more sustained release surfaces layers or regions.

An “adhesive” includes material that chemically binds the adhesive sheet or membrane to the target tissue site or to the expandable member or the cannula to the expandable member. Adhesives can be liquid, semi-solid or in a solid state. The adhesive can be a solvent based adhesive, a polymer dispersion adhesive, a contact adhesive, a pressure sensitive adhesive, a reactive adhesive, such as for example a multi-part adhesive, one part adhesive, heat curing adhesive, moisture curing adhesive, or a combination thereof or the like. The adhesive can be natural or synthetic or a combination thereof. In some embodiments, the adhesive can be disposed or coated on all or portions of the front and/or back of the sheet in a thickness of about 0.1 to about 50 microns.

The term “biodegradable” includes that all or parts of the medical device (e.g., membrane, sheet, drug depot, adhesive agent, etc.) will degrade over time by the action of enzymes, by hydrolytic action and/or by other similar mechanisms in the human body. In various embodiments, “biodegradable” includes that the medical device can break down or degrade within the body to non-toxic components after or while a therapeutic agent has been or is being released. By “bioerodible” it is meant that the medical device will erode or degrade over time due, at least in part, to contact with substances found in the surrounding tissue, fluids or by cellular action. By “bioabsorbable” it is meant that the medical device will be broken down and absorbed within the human body, for example, by a cell or tissue. “Biocompatible” means that the medical device will not cause substantial tissue irritation or necrosis at the target tissue site.

In some embodiments, the medical device (e.g., adhesive sheet, membrane, drug depot) has pores that allow release of the drug from the medical device. The medical device will allow fluid in the device to displace the drug. However, in some embodiments, cell infiltration into the device will be prevented by the size of the pores of the device. In this way, in some embodiments, the medical device should not function as a tissue scaffold and allow tissue growth. Rather, the medical device will be utilized for drug delivery. In some embodiments, the pores in the medical device will be less than 250 to 500 microns. This pore size will prevent cells from infiltrating the medical device and laying down scaffolding cells. Thus, in this embodiment, drug will elute from the medical device as fluid enters the device, but cells will be prevented from entering. Pores can be made using, for example a pore forming agent including polyhydroxy compounds such as a carbohydrate, a polyhydroxy aldehyde, a polyhydroxy ketone, a glycogen, an aldose, a sugar, a mono- or polysaccharide, an oligosaccharide, a polyhydroxy carboxylic compound, polyhydroxy ester compound, a cyclodextrin, a polyethylene glycol polymer, a glycerol an alginate, a chitosan, a polypropylene glycol polymer, a polyoxyethylene-polyoxypropylene block co-polymer, agar, or hyaluronic acid or polyhydroxy derivative compounds, hydroxypropyl cellulose, tween, sorbitan, sorbitan monolaurate, sorbitan monopalmitate, sorbitan monostearate, sorbitan tristearate, sorbitan monooleate, or a combination thereof. In some embodiments, where there are little or no pores, the drug will elute out from the drug depot by the action of enzymes, by hydrolytic action and/or by other similar mechanisms in the human body.

The phrases “sustained release” and “sustain release” (also referred to as extended release or controlled release) are used herein to refer to one or more therapeutic agent(s) that is introduced into the body of a human or other mammal and continuously or continually releases a stream of one or more therapeutic agents over a predetermined time period and at a therapeutic level sufficient to achieve a desired therapeutic effect throughout the predetermined time period. Reference to a continuous or continual release stream is intended to encompass release that occurs as the result of biodegradation in vivo of the medical device (e.g., membrane), or a matrix or component thereof, or as the result of metabolic transformation or dissolution of the therapeutic agent(s) or conjugates of therapeutic agent(s). In some embodiments the medical device (e.g., membrane, adhesive sheet) can have one or more sustained release surface(s), region(s) or layer(s).

The phrase “immediate release” is used herein to refer to one or more therapeutic agent(s) that is introduced into the body and that is allowed to dissolve in or become absorbed at the location to which it is administered, with no intention of delaying or prolonging the dissolution or absorption of the drug. In some embodiments the medical device (e.g., adhesive sheet, membrane, depot) can have one or more immediate release surface(s), regions(s) or layer(s).

The two types of formulations (sustain release and immediate release) may be used in conjunction. The sustained release and immediate release may be in one or more of the same medical device (e.g., adhesive sheet). In various embodiments, the sustained release and immediate release may be part of separate medical devices. For example a bolus or immediate release formulation of analgesic and/or anti-inflammatory agent may be placed at or near the target site and a sustain release formulation may also be placed at or near the same site. Thus, even after the bolus becomes completely accessible, the sustain release formulation would continue to provide the active ingredient for the intended tissue.

In various embodiments, the medical device can be designed to cause an initial burst dose of therapeutic agent within the first twenty-four, forty-eight hours, or seventy-two hours after implantation. “Initial burst” or “burst effect” or “bolus dose” refers to the release of therapeutic agent from the medical device (e.g., one or more surfaces, regions or layers of the adhesive sheet, membrane) during the first twenty-four hours, or forty-eight or seventy-two hours after the device comes in contact with an aqueous fluid (e.g., synovial fluid, cerebral spinal fluid, wound fluid, saline, blood etc.). In some embodiments, the medical device releases 5%, 10%, 15%, 20%, 25%, 30%, 35%, 40%, 45%, or 50% of the total weight of the therapeutic agent loaded in the medical device within the first twenty-four, forty-eight hours, or seventy-two hours after implantation when the device comes into contact with bodily fluid. The “burst effect” or “bolus dose” is believed to be due to the increased release of therapeutic agent from the device (e.g., adhesive sheet, membrane, drug depot). In alternative embodiments, the medical device (e.g., adhesive sheet, membrane, drug depot) is designed to avoid or reduce this initial burst effect (e.g., by applying an outer polymer coating to the sheet, membrane or drug depot or imbedding the drug deep within the polymer, using a polymer having a high molecular weight or combinations thereof, or imbedding drug deep within the adhesive, etc.).

“Analgesic” refers to an agent or compound that can reduce, relieve or eliminate pain. Examples of analgesic agents include but are not limited to acetaminophen, a local anesthetic, such as for example, lidocaine, bupivicaine, ropivacaine, clonidine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, levorphanol, mepiridine, methadone, morphine, nalbuphine, opium, oxycodone, papavereturn, pentazocine, pethidine, phenoperidine, piritramide, dextropropoxyphene, remifentanil, sufentanil, tilidine, tramadol, codeine, dihydrocodeine, meptazinol, dezocine, eptazocine, flupirtine or a combination thereof.

The phrase “anti-inflammatory agent” refers to an agent or compound that has anti-inflammatory effects. These agents may remedy pain by reducing inflammation. Examples of anti-inflammatory agents include, but are not limited to, a statin, sulindac, sulfasalazine, naroxyn, diclofenac, indomethacin, ibuprofen, flurbiprofen, ketoprofen, aclofenac, aloxiprin, aproxen, aspirin, diflunisal, fenoprofen, mefenamic acid, naproxen, phenylbutazone, piroxicam, meloxicam, salicylamide, salicylic acid, desoxysulindac, tenoxicam, ketoralac, clonidine, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, allopurinol, oxypurinol, benzydamine hydrochloride, dimefadane, indoxole, intrazole, mimbane hydrochloride, paranylene hydrochloride, tetrydamine, benzindopyrine hydrochloride, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, triflumidate, fenamates (mefenamic acid, meclofenamic acid), nabumetone, celecoxib, etodolac, nimesulide, apazone, gold, tepoxalin; dithiocarbamate, or a combination thereof. Anti-inflammatory agents also include other compounds such as steroids, such as for example, fluocinolone, cortisol, cortisone, hydrocortisone, fludrocortisone, prednisone, prednisolone, methylprednisolone, triamcinolone, betamethasone, dexamethasone, beclomethasone, fluticasone interleukin-1 receptor antagonists, thalidomide (a TNF-α release inhibitor), thalidomide analogues (which reduce TNF-α production by macrophages), bone morphogenetic protein (BMP) type 2 or BMP-4 (inhibitors of caspase 8, a TNF-α activator), quinapril (an inhibitor of angiotensin II, which upregulates TNF-α), interferons such as IL-11 (which modulate TNF-α receptor expression), and aurin-tricarboxylic acid (which inhibits TNF-α), guanidinoethyldisulfide, or a combination thereof.

Exemplary anti-inflammatory agents include, for example, naproxen; diclofenac; celecoxib; sulindac; diflunisal; piroxicam; indomethacin; etodolac; meloxicam; ibuprofen; ketoprofen; r-flurbiprofen; mefenamic; nabumetone; tolmetin, and sodium salts of each of the foregoing; ketorolac bromethamine; ketorolac tromethamine; ketorolac acid; choline magnesium trisalicylate; rofecoxib; valdecoxib; lumiracoxib; etoricoxib; aspirin; salicylic acid and its sodium salt; salicylate esters of alpha, beta, gamma-tocopherols and tocotrienols (and all their d, l, and racemic isomers); methyl, ethyl, propyl, isopropyl, n-butyl, sec-butyl, t-butyl, esters of acetylsalicylic acid; tenoxicam; aceclofenac; nimesulide; nepafenac; amfenac; bromfenac; flufenamate; phenylbutazone, or a combination thereof.

Exemplary steroids include, for example, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, dexamethasone 21-acetate, dexamethasone 21-phosphate di-Na salt, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide or a combination thereof.

Examples of a useful statin for treatment of pain and/or inflammation include, but are not limited to, atorvastatin, simvastatin, pravastatin, cerivastatin, mevastatin (see U.S. Pat. No. 3,883,140, the entire disclosure is herein incorporated by reference), velostatin (also called synvinolin; see U.S. Pat. Nos. 4,448,784 and 4,450,171 these entire disclosures are herein incorporated by reference), fluvastatin, lovastatin, rosuvastatin and fluindostatin (Sandoz XU-62-320), dalvastain (EP Appln. Publn. No. 738510 A2, the entire disclosure is herein incorporated by reference), eptastatin, pitavastatin, or pharmaceutically acceptable salts thereof or a combination thereof. In various embodiments, the statin may comprise mixtures of (+)R and (−)—S enantiomers of the statin. In various embodiments, the statin may comprise a 1:1 racemic mixture of the statin. Anti-inflammatory agents also include those with anti-inflammatory properties, such as, for example, amitriptyline, carbamazepine, gabapentin, pregabalin, clonidine, or a combination thereof.

Unless otherwise specified or apparent from context, where this specification and the set of claims that follows refer to a drug (e.g., an anti-inflammatory agent, analgesic, and the like) the inventor(s) are also referring to a pharmaceutically acceptable salt of the drug including stereoisomers. Pharmaceutically acceptable salts include those salt-forming acids and bases that do not substantially increase the toxicity of the compound. Some examples of potentially suitable salts include salts of alkali metals such as magnesium, calcium, sodium, potassium and ammonium, salts of mineral acids such as hydrochloric, hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g., p-toluenesulfonic acids, or the like.

“Treating” or treatment of a disease or condition refers to executing a protocol, which may include administering one or more drugs to a patient (human, normal or otherwise, or other mammal), in an effort to alleviate signs or symptoms of the disease. Alleviation can occur prior to signs or symptoms of the disease or condition appearing, as well as after their appearance. Thus, “treating” or “treatment” includes “preventing” or “prevention” of disease or undesirable condition. In addition, “treating” or “treatment” does not require complete alleviation of signs or symptoms, does not require a cure, and specifically includes protocols that have only a marginal effect on the patient. “Reducing pain” includes a decrease in pain and does not require complete alleviation of pain signs or symptoms, and does not require a cure. In various embodiments, reducing pain includes even a marginal decrease in pain. By way of example, the administration of the effective dosages of at least one analgesic agent and at least one anti-inflammatory agent may be used to prevent, treat or relieve the symptoms of pain and/or inflammation.

“Localized” delivery includes delivery where one or more drugs are deposited within a tissue, for example, a nerve root of the nervous system or a region of the brain, or in close proximity (within about 10 cm, or preferably within about 5 cm, for example) thereto. A “targeted delivery system” provides delivery of one or more sheets or membranes having a quantity of therapeutic agent that can be deposited at or near the target site as needed for treatment of pain, inflammation or other disease or condition.

The term “mammal” refers to organisms from the taxonomy class “mammalian,” including but not limited to humans, other primates such as chimpanzees, apes, orangutans and monkeys, rats, mice, cats, dogs, cows, horses, etc. In various embodiments, the mammal is a human patient.

“Localized” delivery includes delivery where one or more medical devices are deposited within a tissue, for example, dermis, lower dermis, muscle, oil and sweat glands, tendons, ligaments, bone, etc. or in close proximity (within about 0.1 cm, or preferably within about 5 cm, for example) thereto. For example, the medical device containing a drug can deliver a dose of it locally that is 20%, 30%, 40%, 50%, 60%, 70%, 80%, 90%, 95%, 99%, 99.9% or 99.999% less than the oral dosage or IV or IM systemic dose. In turn, systemic side effects, such as for example, liver transaminase elevations, hepatitis, liver failure, myopathy, constipation, etc. may be reduced or eliminated. In some embodiments, the medical device is not delivered to the eye and does not include eye formulations.

The phrase “release rate profile” refers to the percentage of active ingredient that is released over fixed units of time, e.g., mcg/hr, mcg/day, mg/day, 10% per day for ten days, etc. As persons of ordinary skill know, a release rate profile may, but need not, be linear. By way of a non-limiting example, the medical device (e.g., adhesive sheet, membrane) may comprise a ribbon-like fiber that releases the therapeutic agent at or near the wound over a period of time.

The term “solid” is intended to mean a rigid material, while, “semi-solid” is intended to mean a material that has some degree of flexibility, thereby allowing the depot to bend and conform to the surrounding tissue requirements. In some embodiments, the medical device has a sufficient flexibility to allow placement within the target tissue site. In some embodiments, the sheet or membrane may have a modulus of elasticity in the range of about 1×⁻10² to about 2×10⁶ dynes/cm², or 1×10⁵ to about 7×10⁵ dynes/cm², or 2×10⁵ to about 5×10⁵ dynes/cm².

“Targeted delivery system” provides delivery of one or more medical devices (e.g., membrane, sheet) having a quantity of therapeutic agent that can be deposited at or near the target site as needed for treatment of the condition or disease.

In some embodiments, the medical device may comprise DLG. The abbreviation “DLG” refers to poly(DL-lactide-co-glycolide). In some embodiments, the medical device may comprise DL. The abbreviation “DL” refers to poly(DL-lactide). In some embodiments, the medical device may comprise LG. The abbreviation “LG” refers to poly(L-lactide-co-glycolide). In some embodiments, the medical device may comprise CL. The abbreviation “CL” refers to polycaprolactone. In some embodiments, the medical device may comprise DLCL. The abbreviation “DLCL” refers to poly(DL-lactide-co-caprolactone). In some embodiments, the medical device may comprise LCL. The abbreviation “LCL” refers to poly(L-lactide-co-caprolactone). In some embodiments, the medical device may comprise G. The abbreviation “G” refers to polyglycolide. In some embodiments, the medical device may comprise PEG. The abbreviation “PEG” refers to poly(ethylene glycol). In some embodiments, the medical device may comprise PLGA. The abbreviation “PLGA” refers to poly(lactide-co-glycolide) also known as poly(lactic-co-glycolic acid), which are used interchangeably. In some embodiments, the medical device may comprise PLA. The abbreviation “PLA” refers to polylactide. In some embodiments, the medical device may comprise POE. The abbreviation “POE” refers to poly(orthoester).

Reference will now be made in detail to certain embodiments of the invention, examples of which are illustrated in the accompanying drawings. While the invention will be described in conjunction with the illustrated embodiments, it will be understood that they are not intended to limit the invention to those embodiments. On the contrary, the invention is intended to cover all alternatives, modifications, and equivalents that may be included within the invention as defined by the appended claims.

The section headings are not meant to limit the disclosure and one section heading can be combined with other section headings.

### Adhesive Membrane

New medical devices and methods are provided that allow insertion of membranes by the surgeon to easily seal the target tissue site and improve healing in a minimally invasive procedure. In some embodiments, the medical device comprises an expandable member that when partially or completely inflated applies pressure to the membrane and allows it to conform to and seal the target tissue site to improve healing. In some embodiments, the membrane can comprise a sheet containing an adhesive to enhance binding to and seal the target tissue site (e.g., annulus fibrosus of the intervertebral disc). In some embodiments, the membrane can comprise a therapeutic agent to further enhance healing of the target tissue site and/or reduce pain.

In some embodiments, there is a medical device for delivering an adhesive membrane adjacent to a target tissue site, the medical device comprising a cannula defining a longitudinal axis and having a proximal end and a distal end, the proximal end of the cannula having an opening configured to receive a flowable material, the distal end of the cannula connected to an expandable member, the expandable member having an interior configured to receive the flowable material and a surface aligned with and contacting at least a portion of the adhesive membrane, the adhesive membrane configured to conform to a select tissue surface of the target tissue site when in an open position.

In some embodiments, the adhesive can be disposed or coated on all or portions of the front and/or back of the sheet or membrane. In some embodiments, the adhesive can be disposed or coated on all or portions of the front and/or back of the sheet or membrane in a thickness of about 0.1 to about 50 microns. In some embodiments, the sheet or membrane has the adhesive disposed on or in all or at discrete positions on its tissue contact surface facing the target tissue site so when the expandable member expands the contact surface side of the sheet or membrane containing the adhesive will open and contact the target tissue site and as the expandable member expands the sheet or membrane will adhere to the target tissue site, also the pressure generated from the expanding member will aid in adhering the sheet or membrane against the target tissue site and, therefore, seal the target tissue site and keep the sheet or membrane at the target tissue site and prevent it from migrating away from it.

In some embodiments, the adhesive sheet or membrane degrades in about six months. The sheet according to the current application is advantageous primarily in that it bonds effectively to tissue, enabling it to be used in a variety of medical applications. In some embodiments, the sheet exhibits good initial adhesion to the tissue to which it is applied (and may thus be described as “self-adhesive”), and furthermore remains well-adhered to the tissue over a longer timescale so as to provide a seal. Without wishing to be bound by any theory, it is believed that the initial adhesion of the sheet or membrane to the tissue is attributable to electronic bonding of the sheet to the tissue, and this is supplemented or replaced by chemical bonding between the tissue-reactive functional groups of the formulation and the tissue. For example, when the adhesive material has amine or thiol groups, there is bonding between amine and/or thiol groups on the tissue surface and the sheet.

The sheet or membrane exhibits good initial adhesion to the tissue surface, this being believed to be due to Van der Waals forces and/or hydrogen bonding between the sheet and the tissue surface. In some embodiments, on contact with the tissue surface, the sheet becomes hydrated, thereby causing reaction between the sheet and the underlying tissue surface. Such reactions result in high adhesion between the sheet and the tissue surface and provide an effective seal. The sheet may absorb physiological fluids (as a consequence of application onto exuding tissue surfaces), and any additional solutions used to hydrate the sheet following application (such fluids can be commonly used solutions used in surgical irrigation), becoming more compliant and adherent to the tissue surfaces, and thereby will provide an adhesive sealant, hemostatic and/or pneumostatic function, if that effect is desired.

In some embodiments, in addition to the chemical means of bonding, pressure from the expanding member will also help the sheet or membrane adhere to the target tissue site.

The use of the sheet or membrane reduces or eliminates the need for additional means of mechanical attachment to the tissue (e.g., sutures or staples). The sheet is applied to the tissue as a preformed article, rather than being prepared by mixing of materials immediately prior to use. The sheet can be any size, shape and configuration and can be in a film, patch, mesh, or the like form. In some embodiments, the adhesive sheet has an overall thickness of from about 0.01 to about 1 mm or from about 0.015 to about 0.05 mm. In some embodiments, the sheet has a tissue contact surface area that accounts for more than 50% of the overall thickness of the sheet. The tissue contact surface of the sheet or membrane can have surface configurations to enhance the seal of the target tissue site such as, for example, rough, arcuate, undulating, dimpled, and/or textured surfaces. In some embodiments, the tissue contact surface of the membrane or sheet is non-porous or substantially non-porous so as to provide an effective seal. However, in some embodiments, the sheet or membrane can degrade over time and will become porous as the membrane or sheet degrades. In addition, in some embodiments, portions of the membrane or sheet can contain a therapeutic agent and be more porous as the seal in these portions of the sheet or membrane is less desired.

In some embodiments, it may be necessary or desirable to incorporate into the sheet a scaffold to increase the mechanical strength and/or flexibility of the film for a particular application. Thus, in some embodiments, there is provided an adhesive sheet comprising a homogenous, pre-formed and cross-linked matrix applied to a scaffold material. Suitable materials for the matrix include, for example, one or more poly (alpha-hydroxy acids), poly (lactide-co-glycolide) (PLGA), polylactide (PLA), poly(L-lactide), polyglycolide (PG), polyglycolic acid (PGA), polyethylene glycol (PEG) conjugates of poly (alpha-hydroxy acids), polyorthoesters (POE), polyaspirins, polyphosphagenes, collagen, hydrolyzed collagen, gelatin, hydrolyzed gelatin, fractions of hydrolyzed gelatin, elastin, starch, pre-gelatinized starch, hyaluronic acid, chitosan, alginate, albumin, fibrin, vitamin E analogs, such as alpha tocopheryl acetate, d-alpha tocopheryl succinate, D,L-lactide, or L-lactide, caprolactone, dextrans, vinylpyrrolidone, polyvinyl alcohol (PVA), PVA-g-PLGA, PEGT-PBT copolymer (polyactive), methacrylates, poly (N-isopropylacrylamide), PEO-PPO-PEO (pluronics), PEO-PPO-PAA copolymers, PLGA-PEO-PLGA, PEG-PLG, PLA-PLGA, poloxamer 407, PEG-PLGA-PEG triblock copolymers, POE, SAIB (sucrose acetate isobutyrate), polydioxanone, methylmethacrylate (MMA), MMA and N-vinylpyyrolidone, polyamide, oxycellulose, copolymer of glycolic acid and trimethylene carbonate, polyesteramides, polyetheretherketone, polymethylmethacrylate, silicone, hyaluronic acid, tyrosine polycarbonate, chitosan, or combinations thereof.

Suitable scaffolds can comprise biocompatible and biodegradable material. The scaffold conveniently has the form of a sheet of material, the homogeneous, pre-formed and cross-linked matrix being applied to one or both sides of the sheet. In such a case, the product has a multilamellar form. The scaffold may be continuous or may be apertured. In some embodiments, the scaffold is perforated. In some embodiments, the scaffold sheet is formed with an array of perforations and the homogenous film is applied to one or both sides of the scaffold sheet.

In some embodiments, the adhesive sheet comprises an adhesive material that binds tissue. The adhesive material may comprise polymers having hydroxyl, carboxyl, and/or amine groups. In some embodiments, polymers having hydroxyl groups include synthetic polysaccharides, such as for example, cellulose derivatives, such as cellulose ethers (e.g., hydroxypropylcellulose). In some embodiments, the synthetic polymers having a carboxyl group, may comprise poly(acrylic acid), poly(methacrylic acid), poly(vinyl pyrrolidone acrylic acid-N-hydroxysuccinimide), and poly(vinyl pyrrolidone-acrylic acid-acrylic acid-N-hydroxysuccinimide) terpolymer. For example, poly(acrylic acid) with a molecular weight greater than 250,000 or 500,000 may exhibit particularly good adhesive performance. In some embodiments, the adhesive can be a polymer having a molecular weight of about 2,000 to about 5,000, or about 10,000 to about 20,000 or about 30,000 to about 40,000.

In some embodiments, the adhesive can comprise imido ester, p-nitrophenyl carbonate, N-hydroxysuccinimide ester, epoxide, isocyanate, acrylate, vinyl sulfone, orthopyridyl-disulfide, maleimide, aldehyde, iodoacetamide or a combination thereof. In some embodiments, the adhesive material can comprise at least one of fibrin, a cyanoacrylate (e.g., N-butyl-2-cyanoacrylate, 2-octyl-cyanoacrylate, etc.), a collagen-based component, a glutaraldehyde glue, a hydrogel, gelatin, an albumin solder, and/or a chitosan adhesives. In some embodiments, the hydrogel comprises acetoacetate esters crosslinked with amino groups or polyethers as mentioned in U.S. Pat. No. 4,708,821. In some embodiments, the adhesive material can comprise poly(hydroxylic) compounds derivatized with acetoacetate groups and/or polyamino compounds derivatized with acetoacetamide groups by themselves or the combination of these compounds crosslinked with an amino-functional crosslinking compounds. In some embodiments, the adhesive comprises one or more of poly (alpha-hydroxy acids), poly (lactide-co-glycolide) (PLGA), polylactide (PLA), polyglycolide (PG), polyethylene glycol (PEG) conjugates of poly (alpha-hydroxy acids), poly(orthoester)s (POE), polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D,L-lactide, or L-lactide, ε-caprolactone, dextrans, vinylpyrrolidone, polyvinyl alcohol (PVA), PVA-g-PLGA, PEGT-PBT copolymer (polyactive), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) poly(lactide-co-glycolide) (PLGA), polylactide (PLA), polyglycolide (PGA), D-lactide, D,L-lactide, L-lactide, D,L-lactide-co-ε-caprolactone, D,L-lactide-co-glycolide-co-ε-caprolactone, poly(D,L-lactide-co-caprolactone), or poly(L-lactide-co-caprolactone), polyester, or copolymers thereof or combinations thereof.

The adhesive can be a solvent based adhesive, a polymer dispersion adhesive, a contact adhesive, a pressure sensitive adhesive, a reactive adhesive, such as for example multi-part adhesives, one part adhesives, heat curing adhesives, moisture curing adhesives, or a combination thereof or the like. The adhesive can be natural or synthetic or a combination thereof.

Contact adhesives are used in strong bonds with high shear-resistance. Pressure sensitive adhesives form a bond by the application of light pressure to bind the adhesive with the target tissue site, cannula and/or expandable member. In some embodiments, to have the device adhere to the target tissue site, pressure is applied in a direction substantially perpendicular to a surgical incision.

Multi-component adhesives harden by mixing two or more components which chemically react. This reaction causes polymers to cross-link into acrylics, urethanes, and/or epoxies. There are several commercial combinations of multi-component adhesives in use in industry. Some of these combinations are: polyester resin-polyurethane resin; polyols-polyurethane resin, acrylic polymers-polyurethane resins or the like. The multi-component resins can be either solvent-based or solvent-less. In some embodiments, the solvents present in the adhesives are a medium for the polyester or the polyurethane resin. Then the solvent is dried during the curing process.

In some embodiments, the adhesive can be a one-part adhesive. One-part adhesives harden via a chemical reaction with an external energy source, such as radiation, heat, and moisture. Ultraviolet (UV) light curing adhesives, also known as light curing materials (LCM), have become popular within the manufacturing sector due to their rapid curing time and strong bond strength. Light curing adhesives are generally acrylic based. The adhesive can be a heat-curing adhesive, where when heat is applied (e.g., body heat), the components react and cross-link. This type of adhesive includes epoxies, urethanes, and/or polyimides. The adhesive can be a moisture curing adhesive that cures when it reacts with moisture present (e.g., bodily fluid) on the substrate surface or in the air. This type of adhesive includes cyanoacrylates or urethanes. The adhesive can have natural components, such as for example, vegetable matter, starch (dextrin), natural resins or from animals e.g. casein or animal glue. The adhesive can have synthetic components based on elastomers, thermoplastics, emulsions, and/or thermosets including epoxy, polyurethane, cyanoacrylate, or acrylic polymers.

Adhesive sheets and adhesives materials suitable for use in the present application are disclosed in published application US20100297218, U.S. Ser. No. 12/602,468, filed Sep. 19, 2007, published application US20090287313, U.S. Ser. No. 12/509,687, filed Jul. 27, 2009, published application US20090044895, U.S. Ser. No. 12/278,252, filed Feb. 2, 2007, published application US 20090018575, U.S. Ser. No. 12/281,289, filed Mar. 1, 2007 and U.S. Pat. Nos. 6,197,296, 7,727,547 and 6,239,190. These entire disclosures are herein incorporated by reference into the present disclosure. A suitable adhesive sheet is available from Tissuemed Limited, UK and can be modified to hold the drug depots.

In some embodiments, the adhesive material comprises less than 50 wt. %, less than 40 wt. %, less than 30 wt. %, less than 20 wt. %, less than 19 wt. %, less than 18 wt. %, less than 17 wt. %, less than 16 wt. %, less than 15 wt. %, less than 14 wt. %, less than 13 wt. %, less than 12 wt. %, less than 11 wt. %, less than 10 wt. %, less than 9 wt. %, less than 8 wt. %, less than 7 wt. %, less than 6 wt. %, less than 5 wt. %, less than 4 wt. %, less than 3 wt. %, less than 2 wt. %, less than 1 wt. % or less than 0.5 wt. % of the medical device.

In some embodiment, an adhesive is disposed at discrete positions on the expandable member and this adhesive has lower adhesive and cohesive properties than the adhesive disposed on or in the tissue contact surface of the sheet or membrane. In this way, on applying a pulling, twisting, pushing or separation force to the cannula and/or expanding member, the sheet or membrane is detached from the expanding member and the sheet or membrane will seal the target tissue site and the cannula and/or expanding member can be removed from the target tissue site. Thus, in some embodiments, the sheet or membrane can have one or more adhesives on all or at discrete positions on the surfaces that each can have the same or different degrees of adhesiveness and/or cohesiveness.

The adhesive material, and/or adhesive sheet or membrane can be biodegradable and can also contain a therapeutic agent. The therapeutic agent can be in immediate release and sustained release form and disposed in a region or throughout the adhesive sheet.

In some embodiments, the medical device is designed so that the majority of the sheet or membrane or surface area of the sheet or membrane contacts the target tissue site and/or bodily fluid to maximize the seal and optional release of the therapeutic agent from it. In some embodiments, the sheet or membrane has a plurality of holes in it placed above, below, front side, back side at discrete positions of the sheet or membrane so that fluid can contact it and the therapeutic agent can be released from it. For example, a plurality of holes can be placed across the back of the adhesive sheet and when the sheet is placed at the target tissue site and separated from the expandable member, the back holes allow drug to diffuse out of the holes and exert action at or near the target tissue site.

In some embodiments, the sheet may be prepared by dissolving or dispersing the components of the matrix in a suitable solvent, and casting the resulting solution into a suitable mold or onto a suitable plate. This can be followed by drying to remove solvent and curing to achieve the desired degree of cross-linking, if cross-linking is desired. Curing can be promoted by prolonged application of elevated temperatures (typically several hours at temperatures in excess of 60° C.). In some embodiments, the sheet will have a water content of less than 10% w/w, and more commonly less than 5% w/w.

Three-dimensional articles (e.g., plugs, meshes, patches, compartments, pockets, etc.) may similarly be prepared by filling of molds with liquid formulations. Sheets comprising a structural scaffold may be prepared by casting the liquid formulation onto the scaffold, by dipping of the scaffold in the liquid formulation or by spraying the formulations onto the scaffold. If the scaffold is required as a backing on one side of the sheet, it may be added during or after the curing process.

Likewise, coatings may be applied to medical devices by casting the formulation over the device, dipping of the devices in liquid formulations or by spraying the devices with the liquid formulation.

In some embodiments, sheets and other formulations may be made up from the following ingredients in the proportions indicated: synthetic polymer(s) with functional groups of from: preferably 20-80% w/w, more preferably 20-70% w/w, 30-60% w/w or 40-60% w/w; additional synthetic polymer(s): preferably 0-30% w/w, more preferably 0-20% w/w or 5-20% w/w; plasticizer(s): preferably 0-30% w/w, more preferably 10-30% w/w or 10-20% w/w; animated and/or thiolated polymer(s): preferably 0-10% w/w, more preferably 2-8% w/w; and non-adhesive film-forming polymer(s): preferably 0-10% w/w, more preferably 0-5% w/w.

The sheet according to the current application is suitable for application to internal surfaces of the body, e.g., it may be applied to internal surfaces (e.g., spine) such as surfaces of internal organs exposed during surgical procedures, including conventional and minimally invasive surgery. In one embodiment, the sheet comprises an analgesic and/or anti-inflammatory agent that can be used to treat post operative pain.

The adhesive sheet, in some embodiments, can comprise a region where a therapeutic agent can be placed. In some embodiments, the region is configured to receive the drug depot and comprises one or more channels, holes, grooves, slits, loops, and/or bands (all or a portion of which can be biodegradable) and the therapeutic agent can be in a drug depot that can have reciprocating or complementary channels, holes, grooves, slits, loops, and/or bands to fit into the region of the adhesive sheet.

The drug depot releases the therapeutic agent. When referring to therapeutic agent, unless otherwise specified or apparent from context it is understood that the inventor is also referring to pharmaceutically acceptable equivalents or derivatives thereof, such as their pharmaceutically acceptable salts, esters, non-esters, prodrugs or active metabolites. Isomers of all disclosed agents are also encompassed by this disclosure.

Some examples of pharmaceutically acceptable salts include those salt-forming acids and bases that do not substantially increase the toxicity of a compound, such as, salts of alkali metals such as magnesium, potassium and ammonium, salts of mineral acids such as hydriodic, hydrochloric, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g., p-toluenesulfonic acids, or the like.

Further, when referring to therapeutic agent and other active ingredients, they may not only be in the salt form, but also in the base form (e.g., free base). Pharmaceutically acceptable salts of therapeutic agent include salts prepared from pharmaceutically acceptable non-toxic bases or acids including inorganic or organic bases, inorganic or organic acids and fatty acids. Salts derived from inorganic bases include aluminum, ammonium, calcium, copper, ferric, ferrous, lithium, magnesium, manganic salts, manganous, potassium, sodium, zinc, and the like. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethyl amine, tripropylamine, tromethamine, or the like.

When the compound of the current application is basic, salts may be prepared from pharmaceutically acceptable non-toxic acids, including inorganic and organic acids. Such acids include acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, trifluoroacetic acid, or the like. Fatty acid salts may also be used, eg., fatty acid salts having greater than 2 carbons, greater than 8 carbons or greater than 16 carbons, such as butyric, caprioc, caprylic, capric, lauric, mystiric, palmitic, stearic, arachidic or the like.

In some embodiments, the therapeutic agent can be in esterified forms, non-esterified forms or a combination thereof.

The loading of the therapeutic agent in the medical device (e.g., in percent by weight relative to the weight of the basic structure) can vary over a wide range, depending on the specific application, and can be determined specifically for the particular case. In some embodiments, the therapeutic agent is in the medical device (e.g., sheet or membrane) in an amount from about 0.1 wt. % to about 50 wt. %, or about 1 wt. % to about 30 wt. %, or about 2.5 wt. % to about 25 wt. %, or about 5 wt. % to about 25 wt. %, or about 10 wt. % to about 20 wt. %, or about 5 wt. % to about 15 wt. %, 5 wt. % to about 10 wt. % based on the total weight of the medical device.

In some embodiments, there is a higher loading of therapeutic agent, e.g., at least 20 wt. %, at least 30 wt. %, at least 40 wt. %, at least 50 wt. %, at least 60 wt. %, at least 70 wt. %, at least 80 wt. %, or at least 90 wt. %.

In some embodiments, the dosage of therapeutic agent may be from approximately 0.0005 to approximately 500 mg/day. In some embodiments, the amount of therapeutic agent is between 1 mg, 2 mg, 3 mg, 4 mg, 5 mg, 6 mg, 7 mg, 8 mg, 9 mg and 10 mg/day. Additional dosages of therapeutic agent include from approximately 0.0005 to approximately 50 μg/day; approximately 0.0005 to approximately 25 μg/day; approximately 0.0005 to approximately 10 μg/day; approximately 0.0005 to approximately 5 μg/day; approximately 0.0005 to approximately 1 μg/day; approximately 0.0005 to approximately 0.75 μg/day; approximately 0.0005 to approximately 0.5 μg/day; approximately 0.0005 to approximately 0.25 μg/day; approximately 0.0005 to approximately 0.1 μg/day; approximately 0.0005 to approximately 0.075 μg/day; approximately 0.0005 to approximately 0.05 μg/day; approximately 0.001 to approximately 0.025 μg/day; approximately 0.001 to approximately 0.01 μg/day; approximately 0.001 to approximately 0.0075 μg/day; approximately 0.001 to approximately 0.005 μg/day; approximately 0.001 to approximately 0.025 μg/day; and approximately 0.002 μg/day. In another embodiment, the dosage of therapeutic agent is from approximately 0.001 to approximately 15 μg/day. In another embodiment, the dosage of therapeutic agent is from approximately 0.001 to approximately 10 ng/day. In another embodiment, the dosage of therapeutic agent is from approximately 0.001 to approximately 5 ng/day. In another embodiment, the dosage of therapeutic agent is from approximately 0.001 to 2.5 ng/day. In some embodiments, the amount of therapeutic agent is between 200 ng/day and 400 ng/day.

The average molecular weight of the polymer of the sheet or membrane can be from about 1000 to about 10,000,000; or about 1,000 to about 1,000,000; or about 5,000 to about 500,000; or about 10,000 to about 100,000 or about 125,000; or about 20,000 to 50,000 daltons.

In various embodiments, the polymer of the sheet or membrane has a molecular weight, as shown by the inherent viscosity (IV), from about 0.10 dL/g to about 1.2 dL/g or from about 0.10 dL/g to about 0.40 dL/g. Other IV ranges include but are not limited to about 0.05 to about 0.15 dL/g, about 0.10 to about 0.20 dL/g, about 0.15 to about 0.25 dL/g, about 0.20 to about 0.30 dL/g, about 0.25 to about 0.35 dL/g, about 0.30 to about 0.35 dL/g, about 0.35 to about 0.45 dL/g, about 0.40 to about 0.45 dL/g, about 0.45 to about 0.50 dL/g, about 0.50 to about 0.70 dL/g, about 0.60 to about 0.80 dL/g, about 0.70 to about 0.90 dL/g, and about 0.80 to about 1.00 dL/g.

The particle size of the therapeutic agent in the sheet (e.g., clonidine) can be from about 1 to about 25 micrometers, or about 5 to 30 or 50 micrometers, however, in various embodiments ranges from about 1 micron to 250 microns may be used.

The therapeutic agent or its pharmaceutically acceptable salt, esters and non-esters thereof may be administered with a muscle relaxant. Exemplary muscle relaxants include by way of example and not limitation, alcuronium chloride, atracurium bescylate, carbamate, carbolonium, carisoprodol, chlorphenesin, chlorzoxazone, cyclobenzaprine, dantrolene, decamethonium bromide, fazadinium, gallamine triethiodide, hexafluorenium, meladrazine, mephensin, metaxalone, methocarbamol, metocurine iodide, pancuronium, pridinol mesylate, styramate, suxamethonium, suxethonium, thiocolchicoside, tizanidine, tolperisone, tubocuarine, vecuronium, or combinations thereof.

The medical device (e.g., sheet or membrane) may comprise additional therapeutic agents. These additional therapeutic agents, in various embodiments, block the transcription or translation of TNF-α or other proteins in the inflammation cascade. Suitable therapeutic agents include, but are not limited to, integrin antagonists, alpha-4 beta-7 integrin antagonists, cell adhesion inhibitors, interferon gamma antagonists, CTLA4-Ig agonists/antagonists (BMS-188667), CD40 ligand antagonists, Humanized anti-IL-6 mAb (MRA, Tocilizumab, Chugai), HMGB-1 mAb (Critical Therapeutics Inc.), anti-IL2R antibodies (daclizumab, basilicimab), ABX (anti IL-8 antibodies), recombinant human IL-10, or HuMax IL-15 (anti-IL 15 antibodies).

Other suitable therapeutic agents include IL-1 inhibitors, such Kineret® (anakinra) which is a recombinant, non-glycosylated form of the human inerleukin-1 receptor antagonist (IL-1Ra), or AMG 108, which is a monoclonal antibody that blocks the action of IL-1. Therapeutic agents also include excitatory amino acids such as glutamate and aspartate, antagonists or inhibitors of glutamate binding to NMDA receptors, AMPA receptors, and/or kainate receptors. Interleukin-1 receptor antagonists, thalidomide (a TNF-α release inhibitor), thalidomide analogues (which reduce TNF-α production by macrophages), bone morphogenetic protein (BMP) type 2 and BMP-4 (inhibitors of caspase 8, a TNF-α activator), quinapril (an inhibitor of angiotensin II, which upregulates TNF-α), interferons such as IL-11 (which modulate TNF-α receptor expression), and aurin-tricarboxylic acid (which inhibits TNF-α), may also be useful as therapeutic agents for reducing inflammation. It is further contemplated that where desirable a pegylated form of the above may be used. Examples of still other therapeutic agents include NF kappa B inhibitors such as glucocorticoids, antioxidants, such as dithiocarbamate, and other compounds, such as, for example, sulfasalazine.

Examples of therapeutic agents suitable for use also include, but are not limited to an anti-inflammatory agent, an analgesic agent, or an osteoinductive growth factor or a combination thereof. Anti-inflammatory agents include, but are not limited to, apazone, celecoxib, diclofenac, diflunisal, enolic acids (piroxicam, meloxicam), etodolac, fenamates (mefenamic acid, meclofenamic acid), gold, ibuprofen, indomethacin, ketoprofen, ketorolac, nabumetone, naproxen, nimesulide, salicylates, sulfasalazine [2-hydroxy-5-[-4-[C2-pyridinylamino)sulfonyl]azo]benzoic acid, sulindac, tepoxalin or tolmetin; as well as antioxidants, such as dithiocarbamate, steroids, such as fluocinolone, cortisol, cortisone, hydrocortisone, fludrocortisone, prednisone, prednisolone, methylprednisolone, triamcinolone, betamethasone, dexamethasone, beclomethasone, fluticasone or a combination thereof.

Suitable analgesic agents include, but are not limited to, acetaminophen, bupivacaine, lidocaine, opioid analgesics such as buprenorphine, butorphanol, dextromoramide, dezocine, dextropropoxyphene, diamorphine, fentanyl, alfentanil, sufentanil, hydrocodone, hydromorphone, ketobemidone, levomethadyl, mepiridine, methadone, morphine, nalbuphine, opium, oxycodone, papavereturn, pentazocine, pethidine, phenoperidine, piritramide, dextropropoxyphene, remifentanil, tilidine, tramadol, codeine, dihydrocodeine, meptazinol, dezocine, eptazocine, flupirtine, amitriptyline, carbamazepine, gabapentin, pregabalin, or a combination thereof.

The therapeutic agent in the device may include, but is not limited to, members of the fibroblast growth factor family, including acidic and basic fibroblast growth factor (FGF-1 and FGF-2) and FGF-4, members of the platelet-derived growth factor (PDGF) family, including PDGF-AB, PDGF-BB and PDGF-AA; EGFs; the TGF-β superfamily, including TGF-β1, 2 or 3; osteoid-inducing factor (OIF); angiogenin(s); endothelins; hepatocyte growth factor or keratinocyte growth factor; members of the bone morphogenetic proteins (BMP's) BMP-1, BMP-3, BMP-2; OP-1, BMP-2A, BMP-2B, or BMP-7; HBGF-1 or HBGF-2; growth differentiation factors (GDF's); members of the hedgehog family of proteins, including indian, sonic and desert hedgehog; ADMP-1; other members of the interleukin (IL) family; or members of the colony-stimulating factor (CSF) family, including CSF-1, G-CSF, and GM-CSF, or isoforms thereof; or VEGF, NELL-1 (neural epidermal growth factor-like 1), CD-RAP (cartilage-derived retinoic acid-sensitive protein) or combinations thereof.

In some embodiments, the device comprises osteogenic proteins. Exemplary osteogenic proteins include, but are not limited to, OP-1, OP-2, OP-3, BMP-2, BMP-3, BMP-3b, BMP-4, BMP-5, BMP-6, BMP-9, BMP-10, BMP-11, BMP-12, BMP-13, BMP-14, BMP-15, GDF-1, GDF-2, GDF-3, GDF-5, GDF-6, GDF-7, GDF-8, GDF-9, GDF-10, GDF-11, GDF-12, CDMP-1, CDMP-2, CDMP-3, DPP, Vg-1, Vgr-1, 60A protein, NODAL, UNIVIN, SCREW, ADMP, NEURAL, and TGF-beta. As used herein, the terms “morphogen,” “bone morphogen,” “BMP,” “osteogenic protein” and “osteogenic factor” embrace the class of proteins typified by human osteogenic protein 1 (hOP-1).

Exemplary growth factors include, but are not limited to, members of the transforming growth factor beta family, including bone morphogenetic protein 2 (BMP-2); bone morphogenetic protein 4 (BMP-4); and transforming growth factors beta-1, beta-2, and beta-3 (potent keratinocyte growth factors). Other useful members of the transforming growth factor beta family include BMP-3, BMP-5, BMP-6, BMP-9, DPP, Vgl, Vgr, 60A protein, GDF-1, GDF-3, GDF-5, GDF-6, GDF-7, CDMP-1, CDMP-2, CDMP-3, BMP-10, BMP-11, BMP-13, BMP-15, Univin, Nodal, Screw, ADMP, Neural, and amino acid sequence variants thereof. Other growth factors include epidermal growth factor (EGF), which induces proliferation of both mesodermal and ectodermal cells, particularly keratinocytes and fibroblasts; platelet-derived growth factor (PDGF), which exerts proliferative effects on mesenchymal cells; fibroblast growth factor (FGF), both acidic and basic; and insulin-like growth factor 1 (IGF-1) or 2 (IGF-2), which mediate the response to growth hormone, particularly in bone growth. Further growth factors include osteogenic proteins. A particularly preferred osteogenic protein is OP-1, also known as bone morphogenetic protein 7 (BMP-7). OP-1 is a member of the transforming growth factor beta gene superfamily.

The therapeutic agent may also be administered with non-active ingredients and they may be in the device with the therapeutic agent. These non-active ingredients may have multi-functional purposes including the carrying, binders, stabilizing, pore forming agents, and/or plasticizers controlling the release of the therapeutic agent(s). Plasticizers include polyhydroxy compounds such as a carbohydrate, a polyhydroxy aldehyde, a polyhydroxy ketone, a glycogen, an aldose, a sugar, a mono- or polysaccharide, an oligosaccharide, a polyhydroxy carboxylic compound, polyhydroxy ester compound, a cyclodextrin, a polyethylene glycol polymer, a glycerol an alginate, a chitosan, a polypropylene glycol polymer, a polyoxyethylene-polyoxypropylene block co-polymer, agar, or hyaluronic acid or polyhydroxy derivative compounds, hydroxypropyl cellulose, tween, sorbitan, sorbitan monolaurate, sorbitan monopalmitate, sorbitan monostearate, sorbitan tristearate, sorbitan monooleate, or a combination thereof.

Exemplary excipients that may be formulated with the therapeutic agent in addition to the biodegradable polymer include but are not limited to MgO (e.g., 1 wt. %), 5050 DLG 6E (Surmodics Pharmaceuticals, Birmingham, Ala.), 5050 DLG 1A (Surmodics Pharmaceuticals, Birmingham, Ala.), mPEG, TBO-Ac, mPEG, Span-65, Span-85, pluronic F127, TBO-Ac, sorbitol, cyclodextrin, maltodextrin, pluronic F68, CaCl, mannitol, trehalose, and combinations thereof. In some embodiments, the excipients comprise from about 0.001 wt. % to about 50 wt. % of the formulation. In some embodiments, the excipients comprise from about 0.001 wt. % to about 40 wt. % of the formulation. In some embodiments, the excipients comprise from about 0.001 wt. % to about 30 wt. % of the formulation. In some embodiments, the excipients comprise from about 0.001 wt. % to about 20 wt. % of the formulation. In some embodiments, the excipients comprise from about 0.001 wt. % to about 10 wt. % of the formulation. In some embodiments, the excipients comprise from about 0.001 wt. % to about 5 wt. % of the formulation. In some embodiments, the excipients comprise from about 0.001 wt. % to about 2 wt. % of the formulation.

In various embodiments, the non-active ingredients will be durable within the tissue site for a period of time equal to or greater than (for biodegradable components) or greater than (for non-biodegradable components) the planned period of drug delivery.

In some instances, it may be desirable to avoid having to remove the membrane or sheet after use. In those instances, the membrane or sheet may comprise a biodegradable material. There are numerous materials available for this purpose and having the characteristic of being able to breakdown or disintegrate over a prolonged period of time when positioned at or near the target tissue. As a function of the chemistry of the biodegradable material, the mechanism of the degradation process can be hydrolytical or enzymatical in nature, or both. In various embodiments, the degradation can occur either at the surface (heterogeneous or surface erosion) or uniformly throughout the drug delivery system depot (homogeneous or bulk erosion).

In various embodiments, the sheet or membrane may comprise a bioerodible, a bioabsorbable, and/or a biodegradable biopolymer that may provide immediate release, or sustained release of the therapeutic agent. Examples of suitable sustained release biopolymers include but are not limited to poly (alpha-hydroxy acids), poly (lactide-co-glycolide) (PLGA), polylactide (PLA), polyglycolide (PG), polyethylene glycol (PEG) conjugates of poly (alpha-hydroxy acids), poly(orthoester)s (POE), polyaspirins, polyphosphagenes, collagen, starch, pre-gelatinized starch, hyaluronic acid, chitosans, gelatin, alginates, albumin, fibrin, vitamin E analogs, such as alpha tocopheryl acetate, d-alpha tocopheryl succinate, D,L-lactide, or L-lactide, -caprolactone, dextrans, vinylpyrrolidone, polyvinyl alcohol (PVA), PVA-g-PLGA, PEGT-PBT copolymer (polyactive), PEO-PPO-PAA copolymers, PLGA-PEO-PLGA, PEG-PLG, PLA-PLGA, poloxamer 407, PEG-PLGA-PEG triblock copolymers, SAIB (sucrose acetate isobutyrate) poly(lactide-co-glycolide) (PLGA), polylactide (PLA), polyglycolide (PGA), D-lactide, D,L-lactide, L-lactide, D,L-lactide-co-ε-caprolactone, D,L-lactide-co-glycolide-co-ε-caprolactone, poly(D,L-lactide-co-caprolactone), or poly(L-lactide-co-caprolactone), or copolymers thereof or combinations thereof PEG may be used as a plasticizer for PLGA, but other polymers/excipients may be used to achieve the same effect. PEG imparts malleability to the resulting formulations. In some embodiments, these biopolymers may also be coated on the sheet or membrane to provide the desired release profile. In some embodiments, the coating thickness may be thin, for example, from about 5, 10, 15, 20, 25, 30, 35, 40, 45 or 50 microns to thicker coatings 60, 65, 70, 75, 80, 85, 90, 95, 100 microns to delay release of the drug from the sheet or membrane. In some embodiments, the range of the coating on the sheet or membrane ranges from about 5 microns to about 250 microns or 5 microns to about 200 microns to delay release from the sheet or membrane.

In various embodiments, the sheet or membrane comprises poly(lactide-co-glycolide) (PLGA), polylactide (PLA), polyglycolide (PGA), D-lactide, D,L-lactide, L-lactide, D,L-lactide-co-ε-caprolactone, D,L-lactide-co-glycolide-co-ε-caprolactone, poly(lactide-co-glycolide) (PLGA), polylactide (PLA), polyglycolide (PGA), D-lactide, D,L-lactide, L-lactide, D,L-lactide-co-ε-caprolactone, D,L-lactide-co-glycolide-co-e-caprolactone, poly(D,L-lactide-co-caprolactone), or poly(L-lactide-co-caprolactone), or copolymers thereof or a combination thereof.

In some embodiments, the sheet or membrane comprises one or more polymers (e.g., PLA, PLGA, etc.) having a MW of from about 15,000 to about 150,000 Da or from about 25,000 to about 100,000 Da.

The sheet or membrane may optionally contain inactive materials such as buffering agents and pH adjusting agents such as potassium bicarbonate, potassium carbonate, potassium hydroxide, sodium acetate, sodium borate, sodium bicarbonate, sodium carbonate, sodium hydroxide or sodium phosphate; degradation/release modifiers; drug release adjusting agents; emulsifiers; preservatives such as benzalkonium chloride, chlorobutanol, phenylmercuric acetate and phenylmercuric nitrate, sodium bisulfate, sodium bisulfate, sodium thiosulfate, thimerosal, methylparaben, polyvinyl alcohol and phenylethyl alcohol; solubility adjusting agents; stabilizers; and/or cohesion modifiers. If the sheet or membrane is to be placed in the spinal area, in various embodiments, the sheet or membrane may comprise sterile preservative free material.

The sheet or membrane can be different sizes, shapes and configurations. There are several factors that can be taken into consideration in determining the size, shape and configuration of the sheet or membrane. For example, both the size and shape may allow for ease in positioning the sheet or membrane at the target tissue site that is selected as the implantation or injection site. In addition, the shape and size of the system should be selected so as to minimize or prevent the sheet or membrane from moving after implantation or injection. In various embodiments, the sheet or membrane can be shaped like a sphere, a cylinder such as a rod or fiber, a flat surface such as a disc, film or sheet (e.g., ribbon-like), strip, mesh or the like. Flexibility may be a consideration so as to facilitate placement of the sheet or membrane. In some embodiments, the sheet or membrane has a modulus of elasticity (Young's modulus) in the range of about 1×−10² to about 6×10⁵ dynes/cm², or 2×10⁴ to about 5×10⁵ dynes/cm², or 5×10⁴ to about 5×10⁵ dynes/cm².

Radiographic markers can be included on the sheet or membrane to permit the user to position the sheet or membrane accurately into the target site of the patient. These radiographic markers will also permit the user to track movement and degradation of the sheet or membrane at the site over time. In this embodiment, the user may accurately position the sheet or membrane in the site using any of the numerous diagnostic imaging procedures. Such diagnostic imaging procedures include, for example, X-ray imaging or fluoroscopy. Examples of such radiographic markers include, but are not limited to, barium, calcium phosphate, bismuth, iodine, tantalum, tungsten, and/or metal beads or particles. In various embodiments, the radiographic marker could be embedded in the adhesive sheet and could be in a spherical shape or a ring around the sheet or membrane.

Flexibility may be a consideration so as to facilitate placement of the sheet or membrane. In various embodiments, the sheet or membrane can be different sizes, for example, the sheet or membrane may be a length of from about 2 to 4 cm and width of from about 1-2 cm and thickness of from about 0.25 to 1 mm, or length of from about 0.5 mm to 5 cm and have a diameter of from about 0.01 to about 2 mm. In various embodiments, the sheet or membrane is a strip having dimensions of 2.5 cm×1.5 cm×0.5 mm. In various embodiments, the sheet or membrane may have a layer thickness of from about 0.005 to 1.0 mm, such as, for example, from 0.05 to 0.75 mm.

In various embodiments, the sheet or membrane may have an agent to enhance porosity such as, for example, hydroxypropyl cellulose, hydroxypropyl methylcellulose, hydroxyethyl methylcellulose, carboxymethylcellulose and salts thereof, Carbopol, poly-(hydroxyethylmethacrylate), poly-(methoxyethylmethacrylate), poly(methoxyethoxyethyl methacrylate), polymethylmethacrylate (PMMA), methylmethacrylate (MMA), gelatin, polyvinyl alcohols, propylene glycol, mPEG, PEG 200, PEG 300, PEG 400, PEG 500, PEG 600, PEG 700, PEG 800, PEG 900, PEG 1000, PEG 1450, PEG 3350, PEG 4500, PEG 8000 or combinations thereof.

### Expandable Member

The term “expandable member” as used herein includes a component of a medical device that is configured to be changed or moved from a collapsed, deflated, or closed configuration to an expanded, inflated, or open configuration in which the expandable member is larger than in the collapsed configuration. In some variations, the expandable member is configured to be expanded, for example, by introducing a medium such as liquid, powder, and/or gas into the interior of the expandable member. The expandable member can be, for example, a balloon configured to expand from a collapsed configuration to an expanded configuration. In some applications, the balloon is constructed, at least in part, from a low-compliant material.

In some embodiments, for example, an expandable member can be a high-compliant balloon configured to significantly elastically deform when expanded. In other embodiments, an expandable member can be a low-compliant balloon configured to compact and/or displace material without significantly deforming. The compliance of a balloon is the degree to which a size of the balloon in an unfolded state changes as a function of the pressure within the balloon. For example, in some embodiments, the compliance of a balloon can be used to characterize the change in the diameter of the unfolded balloon as a function of the balloon pressure. In some embodiments, the diameter of an unfolded balloon can be characterized as a low-compliant balloon that can change by one to ten percent over the range of inflation pressure. In other embodiments, an unfolded balloon in which the diameter changes by as much as 20 percent may be characterized as a low-compliant balloon. Similarly, in some embodiments, the diameter of an unfolded balloon characterized as a high-compliant balloon can change by 18 to 30 percent. In other embodiments, the diameter of an unfolded high-compliant balloon can change by as much as 100 to 600 percent over the range of inflation.

In some embodiments, the expandable member can expand more in a radial direction than in height. In some embodiments, the expandable member has a diameter in the closed or collapsed position that is the same size or slightly larger than the diameter of the cannula. In some embodiments, the adhesive sheet or membrane is attached to the expandable member by an adhesive. The adhesive can dissociate from the expandable member in a few minutes or after it contacts bodily fluids or upon application of a separation force to the expandable member and/or cannula.

In some embodiments, the adhesive can be disposed at discrete positions between the balloon and the adhesive membrane or sheet so that when fluid contacts the adhesive, the balloon can easily be detached from the adhesive membrane or sheet. In some embodiments, the adhesive can be disposed at discrete positions between the balloon and the adhesive membrane or sheet so that when a turning force, pulling force, or other separation force is applied to the cannula and/or balloon, the adhesive membrane or sheet can be separated from the balloon.

In some embodiments, the compliance of a balloon can be used to characterize the change in the length of the balloon as a function of the balloon pressure. The change in length can also be referred to as the elongation percentage of the balloon. In other embodiments, the compliance of a balloon can be used to characterized the change in volume of the balloon as a function of the balloon pressure. Similarly, in some embodiments, the compliance of a balloon can be used to characterize the material properties from which the balloon or portions of the balloon are constructed.

In some embodiments, for example, an expandable member can be constructed from a low-compliant material (e.g., a material having a low modulus of elasticity) comprising a polymer, such as polyamide, polyethylene terephthalate (PET), Nylons, cross-linked Polyethylene, PEBAX®, Polyurethanes, PVC or any blend of these compounds. In some embodiments, an expandable member can be constructed from Nylon 12.

In some embodiments, a method is provided that includes inserting a distal portion of a cannula containing the expandable member (e.g., balloon) and the adhesive sheet or membrane into a patient's body to establish a percutaneous path to a tissue in the patient's body (e.g., a vertebral body). The balloon will have the adhesive sheet attached to it and in some embodiments, be the same size or larger than the diameter of the catheter. The balloon and/or adhesive sheet can be advanced into the tissue at or near the target tissue site.

Once the expandable member (e.g., balloon) and the adhesive sheet are positioned within the tissue, a fluid is introduced through the proximal end of the cannula and travels along its longitudinal axis and into the interior of the balloon to inflate the balloon. In some embodiments, for example, the pressure of the fluid inside the balloon may need to be maintained below 2 MPa, below 1 MPa, below 0.5 MPa to open or expand the balloon. All or a portion of the sheet or membrane is attached to all or a portion of the exterior surface of the balloon, for example by an adhesive. This in turn will cause the sheet or membrane to unfold or open and the adhesive and/or pressure from the expansion of the balloon will cause the tissue contacting surface of the sheet or membrane to adhere to the target tissue site (e.g., hole in the annulus, or hole in a joint, etc.) via the adhesive and provide a seal over the hole.

After the sheet or membrane is implanted or seals the target tissue site, the expandable member disposed on the distal end of the cannula can be rotated, pulled away from the target tissue site or a separation force applied to it in a controlled and/or incremental fashion to separate the sheet or membrane from the expandable member. In some embodiments, the cannula, balloon and/or sheet or membrane can comprise one or more markers to aid in indicating position of the cannula, balloon and/or sheet or membrane in vivo. After the membrane or sheet seals the target tissue site, the cannula and the expandable member is withdrawn from the patient's body via the cannula.

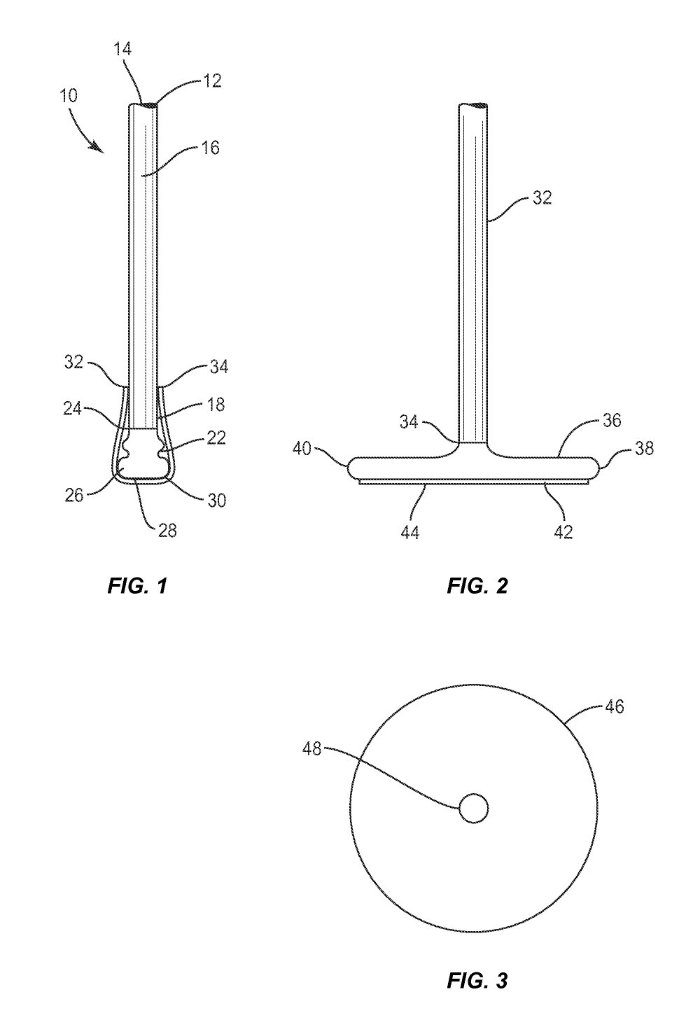

FIG. 1 illustrates a side view of one embodiment of the medical device 10 having an adhesive sheet or membrane containing an adhesive material disposed on one side of the sheet or membrane that comprises a tissue contacting surface 30 that can adhere to and/or seal a target tissue site (e.g., a hole in an annulus) not shown. A second side of the adhesive sheet or membrane has an expandable member contacting surface 28 that comprises an adhesive material at discrete positions on or in the second side. The second side having the expandable member contacting surface 28 attaches to the expandable member 26 (e.g., balloon) via an adhesive material. The adhesive sheet or membrane, in some embodiments, can have the adhesive material disposed uniformly throughout it on one or more of its side or it can have the adhesive material disposed at discrete positions on one or more of its sides. The adhesive material on side 28 can, in some embodiments, have the same or less adhesiveness and/or cohesiveness as the adhesive material that is on the tissue contacting surface 30. In this way, the expandable member 26 is easily detached from the adhesive sheet by a separation force (e.g., twisting, pulling, etc.) after the adhesive sheet or membrane is implanted.

The adhesive sheet or material has opposed unfolding edges 32 and 34 that are substantially parallel to each other. Expandable member 26 is shown in its deflated, closed or collapsed state 22. Once the expandable member (e.g., balloon) and the adhesive sheet are positioned at or near the target tissue site, a flowable material is introduced from the proximal end 12 through opening 14 that can be attached to a delivery device, or opening 14 can have a leur fitting or threading to attach to a syringe. In some embodiments, the proximal end 12 of the cannula 16 can slidably receive a plunger (not shown) that can push gas, liquid, powder or the expandable member 26 out of the distal end 18 of the cannula 16.

The flowable material flows to distal end 18 of the cannula into the interior of the expandable member 26 causing the expandable member to open, expand, or inflate radially causing the adhesive sheet or membrane and edges 32 and 34 to unfold or open as the expandable member inflates. Pressure from the expandable member causes movement of the adhesive sheet or membrane to move away from the cannula and edges 32 and 34 of the adhesive membrane or sheet move counterclockwise and clockwise with respect to each other until the desired inflation of the expandable member is reached, often when the adhesive sheet or membrane is planar or substantially planar to the select target tissue site (e.g., hole in an annulus of an intervertebral disc). In some embodiments, the cannula 16 is perpendicular or substantially perpendicular to the adhesive sheet or membrane after the expandable member is inflated. In some embodiments, the expandable member 26 can be attached to the cannula via an adhesive. In some embodiments, the expandable member can be advanced through the interior of the cannula. In some embodiments, as shown in FIG. 1, the expandable member comprises a port 24 configured to receive the distal end 18 of the cannula. The port 24 can provide a fluid tight seal for the expandable member around the cannula. In some embodiments, the port can comprise elastic material and provide a snug fit around the cannula or, in some embodiments, the port can comprise adhesive material to fluidly couple the expandable member to the cannula.

In some embodiments, the expandable member has a diameter in the closed, deflated or collapsed position that is the same size or slightly larger than the diameter of the cannula as shown in FIG. 1. In some embodiments, the expandable member can be contiguous with the adhesive sheet or membrane. In some embodiments, as shown in FIG. 1, a portion of the expandable member 26, contacts a portion of the adhesive sheet or membrane. In the embodiment shown in FIG. 1, there are gaps or pockets between the cannula and edges 32 and 34 that are substantially parallel to each other. These gaps or pockets are disposed above at least a portion of the expandable member and widen or disappear as the expandable member is inflated and moves radially or laterally with the adhesive sheet or membrane. In this way, when the device 10 has the expandable member inflated, the device acts as a miniaturized surgeons' finger, where it can apply pressure to the target tissue site and the adhesive sheet or membrane will adhere to the site and provide an effective seal for it. The device is particularly useful when working in small and confined tissue areas, such as the tissue area at or near the spine.

Referring to FIG. 2, once the expandable member (e.g., balloon) and the adhesive sheet are positioned within the tissue, a flowable material (e.g., liquid, gas, powder or other flowable material) is introduced through the proximal end of the cannula and travels along its longitudinal axis of the cannula 35 and into the interior of the expandable member (e.g., balloon) to open, expand or inflate it. The expandable member 36 is shown in FIG. 2 in its inflated position. This in turn causes adhesive sheet or membrane 42 to unfold or open so that the tissue contact surface of the adhesive sheet or membrane 42 can adhere to a target tissue site via adhesive 44 disposed on its surface. From FIG. 2, the expandable member 36 comprises a port 37 that seals the cannula to the expandable member. The port 37 can also include an adhesive material or an interference fitting to insure that the cannula 35 is attached to the expandable member 36, before, during and after the adhesive sheet or membrane is opened or unfolded and adheres to and seals the target tissue site.

The adhesive sheet or membrane 42 has an expandable member contacting surface that temporarily adheres to the expandable member 36 by a temporary adhesive (e.g., polymer) that is soluble in bodily fluid and can degrade in vivo in minutes or sooner so as to allow the expandable member 36 to be removed from the adhesive sheet or membrane 42 after it adheres to the target tissue site by applying a separation force (e.g., twisting or pulling force, etc.) to the cannula and/or expandable member so as to separate the adhesive sheet or membrane 42 from the expandable member 36. Therefore, the adhesive sheet or membrane can have adhesives of varied degrees of adhesiveness and/or cohesiveness, where the tissue contact surface of the adhesive sheet or membrane can have a higher degree of adhesiveness and cohesiveness than the expandable member contacting surface of the adhesive sheet or membrane, which can have a temporary adhesive disposed on all or discrete positions of it. In this way, the expandable member can be more easily separated from the adhesive sheet or membrane. In the embodiment shown in FIG. 2, the expandable member can expand or inflate more in a radial direction by points 38 and 40 (that is flat) than in height. This direction is a direction transverse to the cannula 35 to move the adhesive sheet or membrane against the select tissue surface when the adhesive membrane or sheet is in the open position. The adhesive sheet or membrane, in some embodiments, can be flexible so as to conform to the target tissue site.

In some embodiments, the expandable member can be contiguous with the adhesive sheet or membrane 42. In other embodiments, it is longer or smaller than the adhesive sheet or membrane. In some embodiments, the expandable member can have a surface area that is larger, the same size, or smaller than the surface area of the adhesive member or sheet. Although one adhesive sheet or membrane is shown, it will be understood that one, two, three, four, five, six, seven sheets or membranes or more can be attached to the expandable member.

The expandable member can expand or inflate to the necessary diameter to cause the adhesive sheet or membrane to contact the target tissue site. The expandable member can be any shape so long as it is configured to unfold or open the adhesive sheet or membrane so that it can abut the tissue plane and cause it to adhere and/or seal the target tissue site. The expandable member and/or adhesive sheet or membrane can be any shape, for example, conical, square, oval, flat circular, rectangular, spherical, tapered, dog bone, offset, crescent, or the like.

FIG. 3 illustrates a top view of one embodiment of the cannula 46 that can receive fluid material (e.g., gas, liquid, powder) to inflate the expandable member. The cannula is fluidly connected to the expandable member by a port at 48.

The adhesive prevents the sheet or membrane from migrating away from the target tissue site as blood flow or fluid flow in the area increase. In addition, when multiple sheet or membranes are implanted, they can be evenly distributed around the target tissue site (e.g., surgical site) to optimize their clinical efficacy.

In some embodiments, the adhesive can be disposed or coated on all or portions of the front and/or back of the sheet or membrane or on the sheet or membrane itself.

In some embodiments, the medical device is designed that the majority of the sheet or membrane or surface area of the sheet or membrane contacts the target tissue site and/or bodily fluid to maximize release of the therapeutic agent from the sheet or membrane. In some embodiments, the sheet has a plurality of holes in it placed above, below or continuously with the sheet or membrane so that fluid can contact the sheet or membrane and the therapeutic agent can be released from the sheet or membrane.

In some embodiments, the surface area of the adhesive can be 10%, 20%, 30%, 40%, 50%, 60%, 70%, 80%, 90%, 95% or less than the surface area of the sheet or membrane. The adhesive can be disposed throughout the entire surface of the sheet or disposed on portions of the sheet. For example, there can be a portion of the adhesive sheet that does not contain any adhesive and can be a silhouette around each sheet or membrane. In some embodiments, the adhesive of the adhesive sheet is a dry material and the user presses it against a target tissue site (e.g., open surgical wound) for a few seconds to a few minutes, where the fluid from the site will contact the adhesive (which can be dry and then hydrate it) and the adhesive sheet will stick to the site leaving the sheet or membrane exposed to the target tissue site (e.g., open surgical wound).

Surgical procedures can be used to attach the medical device at or near the target tissue site. In such applications, the device is positioned in the desired orientation (e.g., against the tissue plane) at or near the target tissue site with the adhesive material touching the target tissue site so that the adhesive binds to and seals the target tissue site and reduces or inhibits migration of the medical device away from the target tissue site.

The sheet or membrane can now be orientated and placed with pressure at or near the target tissue site where the adhesive on the same or opposite side of the sheet or membrane and the sheet or membrane contacts the target tissue site and the adhesive material holds the sheet or membrane in position so the surface of the sheet or membrane containing the therapeutic agent can be released. The adhesive prevents the sheet or membrane from migrating away from the target tissue site as blood flow or fluid flow in the area increase. In addition, when multiple sheets or membranes are implanted, they can be evenly distributed around the target tissue site (e.g., surgical site) to optimize their clinical efficacy.

In some embodiments, the sheet or membrane can have a sustained release surface that releases the therapeutic agent in a controlled manner over an extended period of time (e.g., 3 days or longer). In some embodiments, the adhesive sheet contains immediate release and/or sustained release formulations of the therapeutic agent. In some embodiments, the adhesive sheet contains no therapeutic agent.

In some embodiments, the medical device is designed that the majority of the sheet or membrane or surface area of the sheet or membrane contacts the target tissue site and/or bodily fluid to maximize release of the therapeutic agent from the sheet or membrane.

In some embodiments, the sheet or membrane releases about 5% to about 45% of an analgesic relative to a total amount of the analgesic loaded in the medical device over a first period of up to 48 hours and about 55% to about 95% of the analgesic relative to a total amount of the analgesic loaded in the medical device over a subsequent period of at least one day.

In some embodiments, the adhesive material can be applied as a coating or film on the sheet or membrane or expandable member. In other embodiments, the adhesive material can be applied as a sheet flat or rolled around the sheet or membrane or expandable member. In some embodiments, the adhesive material has a surface area that is smaller than the surface area of the sheet or membrane.

In some embodiments, one or more regions of the adhesive membrane or sheet is porous to allow fluid in that contacts the sheet or membrane to release the therapeutic agent.

In some embodiments, one or more regions of the adhesive, sheet or membrane, or expandable member surface(s) can comprise a biocompatible lubricant to reduce friction when the sheet or membrane slides into the compartment. Suitable examples of lubricants include, without limitations, hyaluronic acid, hyaluronan, lubricin, polyethylene glycol, or sorbitol, magnesium stearate, calcium stearate, zinc stearate, stearic acid, hydrogenated vegetable oils, talc, mineral oil or any combinations thereof.

It will be understood by those of ordinary skill in the art that the sheet or membrane and/or expandable member can be made from the same or different material and the adhesive can be disposed on all sides, or portions of one or more sides.

In some embodiments, the medical device is suitable for parenteral administration. The term “parenteral” as used herein refers to modes of administration that bypass the gastrointestinal tract, and include for example, intravenous, intramuscular, continuous or intermittent infusion, intraperitoneal, intrasternal, subcutaneous, intra-operatively, intrathecally, intradiscally, peridiscally, epidurally, perispinally, intraarticular injection or combinations thereof. In some embodiments, the injection is intrathecal, which refers to an injection into the spinal canal (intrathecal space surrounding the spinal cord). An injection may also be into a muscle or other tissue.

In some embodiments, a method is provided for treating a target tissue site, the method comprising: inserting a balloon adjacent to the target tissue site, the balloon having a surface aligned with and connected to at least a portion of an adhesive sheet, the balloon configured to move from a deflated position when the adhesive sheet is in a closed position to an inflated position when the adhesive sheet is in an open position; positioning the adhesive sheet adjacent to a select tissue surface of the target tissue site; inflating the balloon to move the adhesive sheet to an open position so as to conform the adhesive sheet to the select tissue surface and adhere the adhesive sheet to the select tissue surface of the target tissue site.

After the adhesive sheet or membrane is deployed, the expandable member can be broken away from the adhesive sheet or membrane, this can be passively or actively by twisting or turning the cannula and/or deflating the expandable member. After the expandable member is deflated or collapsed by withdrawing gas, fluid or powder from it back up the cannula, the cannula and the expandable member are not designed to remain in the body and can be removed from the body and the target tissue site (e.g., nucleus pulposis or annulus fibrosis of an intervertebral disc).

The target tissue site can be the nucleus pulposis or annulus fibrosis of an intervertebral disc. The target tissue site can also be any organ that has fluids or gases that can have a leak such as the dura, blood vessels or lungs. The target tissue site could also be any internal structure that is accessible via a cannula or tube such as the heart, liver, kidneys, etc.

### Method of Making Membrane

In various embodiments, the sheet or membrane comprising the therapeutic agent can be made by combining a biocompatible polymer and a therapeutically effective amount of therapeutic agent or pharmaceutically acceptable salt thereof and forming the implantable sheet or membrane from the combination.

Various techniques are available for forming at least a portion of a sheet or membrane from the biocompatible polymer(s), therapeutic agent(s), and optional materials, including solution processing techniques and/or thermoplastic processing techniques. Where solution processing techniques are used, a solvent system is typically selected that contains one or more solvent species. The solvent system is generally a good solvent for at least one component of interest, for example, biocompatible polymer and/or therapeutic agent. The particular solvent species that make up the solvent system can also be selected based on other characteristics, including drying rate and surface tension.

Solution processing techniques include solvent casting techniques, spin coating techniques, web coating techniques, solvent spraying techniques, dipping techniques, techniques involving coating via mechanical suspension, including air suspension (e.g., fluidized coating), ink jet techniques and electrostatic techniques. Where appropriate, techniques such as those listed above can be repeated or combined to build up the sheet or membrane to obtain the desired release rate and desired thickness.

In various embodiments, a solution containing solvent and biocompatible polymer are combined and placed in a mold of the desired size and shape. In this way, polymeric regions, including barrier layers, lubricious layers, and so forth can be formed. If desired, the solution can further comprise, one or more of the following: a therapeutic agent and other therapeutic agent(s) and other optional additives such as radiographic agent(s), etc. in dissolved or dispersed form. This results in a polymeric matrix region containing these species after solvent removal. In other embodiments, a solution containing solvent with dissolved or dispersed therapeutic agent is applied to a pre-existing polymeric region, which can be formed using a variety of techniques including solution processing and thermoplastic processing techniques, whereupon the therapeutic agent is imbibed into the polymeric region.

Thermoplastic processing techniques for forming the sheet or membrane or portions thereof include molding techniques (for example, injection molding, rotational molding, and so forth), extrusion techniques (for example, extrusion, co-extrusion, multi-layer extrusion, and so forth) and casting.

Thermoplastic processing in accordance with various embodiments comprises mixing or compounding, in one or more stages, the biocompatible polymer(s) and one or more of the following: therapeutic agent, optional additional therapeutic agent(s), radiographic agent(s), and so forth. The resulting mixture is then shaped into an implantable sheet or membrane. The mixing and shaping operations may be performed using any of the conventional devices known in the art for such purposes.

During thermoplastic processing, there exists the potential for the therapeutic agent(s) to degrade, for example, due to elevated temperatures and/or mechanical shear that are associated with such processing. For example, therapeutic agent may undergo substantial degradation under ordinary thermoplastic processing conditions. Hence, processing is preferably performed under modified conditions, which prevent the substantial degradation of the therapeutic agent(s). Although it is understood that some degradation may be unavoidable during thermoplastic processing, degradation is generally limited to 10% or less. Among the processing conditions that may be controlled during processing to avoid substantial degradation of the therapeutic agent(s) are temperature, applied shear rate, applied shear stress, residence time of the mixture containing the therapeutic agent, and the technique by which the polymeric material and the therapeutic agent(s) are mixed.

Mixing or compounding biocompatible polymer with therapeutic agent(s) and any additional additives to form a substantially homogenous mixture thereof may be performed with any device known in the art and conventionally used for mixing polymeric materials with additives.

Where thermoplastic materials are employed, a polymer melt may be formed by heating the biocompatible polymer, which can be mixed with various additives (e.g., therapeutic agent(s), inactive ingredients, etc.) to form a mixture. A common way of doing so is to apply mechanical shear to a mixture of the biocompatible polymer(s) and additive(s). Devices in which the biocompatible polymer(s) and additive(s) may be mixed in this fashion include devices such as single screw extruders, twin screw extruders, banbury mixers, high-speed mixers, ross kettles, and so forth.

Any of the biocompatible polymer(s) and various additives may be premixed prior to a final thermoplastic mixing and shaping process, if desired (e.g., to prevent substantial degradation of the therapeutic agent among other reasons).

For example, in various embodiments, a biocompatible polymer is precompounded with a radiographic agent (e.g., radio-opacifying agent) under conditions of temperature and mechanical shear that would result in substantial degradation of the therapeutic agent, if it were present. This precompounded material is then mixed with therapeutic agent under conditions of lower temperature and mechanical shear, and the resulting mixture is shaped into the sheet or membrane containing the therapeutic agent. Conversely, in another embodiment, the biocompatible polymer can be precompounded with the therapeutic agent under conditions of reduced temperature and mechanical shear. This precompounded material is then mixed with, for example, a radio-opacifying agent, also under conditions of reduced temperature and mechanical shear, and the resulting mixture is shaped into the sheet or membrane.

The conditions used to achieve a mixture of the biocompatible polymer and therapeutic agent and other additives will depend on a number of factors including, for example, the specific biocompatible polymer(s) and additive(s) used, as well as the type of mixing device used.

As an example, different biocompatible polymers will typically soften to facilitate mixing at different temperatures. For instance, where a sheet or membrane is formed comprising PLGA or PLA polymer, a radio-opacifying agent (e.g., bismuth subcarbonate), and a therapeutic agent prone to degradation by heat and/or mechanical shear (e.g., therapeutic agent), in various embodiments, the PGLA or PLA can be premixed with the radio-opacifying agent at temperatures of about, for example, 150° C. to 170° C. The therapeutic agent is then combined with the premixed composition and subjected to further thermoplastic processing at conditions of temperature and mechanical shear that are substantially lower than is typical for PGLA or PLA compositions. For example, where extruders are used, barrel temperature, volumetric output are typically controlled to limit the shear and therefore to prevent substantial degradation of the therapeutic agent(s). For instance, the therapeutic agent and premixed composition can be mixed/compounded using a twin screw extruder at substantially lower temperatures (e.g., 100-105° C.), and using substantially reduced volumetric output (e.g., less than 30% of full capacity, which generally corresponds to a volumetric output of less than 200 cc/min). It is noted that this processing temperature is well below the melting points of therapeutic agent because processing at or above these temperatures will result in substantial therapeutic agent degradation. It is further noted that in certain embodiments, the processing temperature will be below the melting point of all bioactive compounds within the composition, including the therapeutic agent. After compounding, the resulting sheet or membrane is shaped into the desired form, also under conditions of reduced temperature and shear.

In other embodiments, biodegradable polymer(s) and one or more therapeutic agents are premixed using non-thermoplastic techniques. For example, the biocompatible polymer can be dissolved in a solvent system containing one or more solvent species. Any desired agents (for example, a radio-opacifying agent, a therapeutic agent, or both radio-opacifying agent and therapeutic agent) can also be dissolved or dispersed in the solvents system. Solvent is then removed from the resulting solution/dispersion, forming a solid material. The resulting solid material can then be granulated for further thermoplastic processing (for example, extrusion) if desired.

As another example, the therapeutic agent can be dissolved or dispersed in a solvent system, which is then applied to a pre-existing sheet or membrane (the pre-existing sheet or membrane can be formed using a variety of techniques including solution and thermoplastic processing techniques, and it can comprise a variety of additives including a radio-opacifying agent and/or viscosity enhancing agent), whereupon the therapeutic agent is imbibed on or in the sheet or membrane. As above, the resulting solid material can then be granulated for further processing, if desired.

Typically, an extrusion process may be used to form the sheet or membrane comprising a biocompatible polymer(s), therapeutic agent(s) and radio-opacifying agent(s). Co-extrusion may also be employed, which is a shaping process that can be used to produce a sheet or membrane comprising the same or different layers or regions (for example, a structure comprising one or more polymeric matrix layers or regions that have permeability to fluids to allow immediate and/or sustained drug release). Multi-region sheet or membranes can also be formed by other processing and shaping techniques such as co-injection or sequential injection molding technology.

In various embodiments, the sheet or membrane that may emerge from the thermoplastic processing is cooled. Examples of cooling processes include air cooling and/or immersion in a cooling bath. In some embodiments, a water bath is used to cool the extruded sheet or membrane. However, where a water-soluble therapeutic agent such as therapeutic agent are used, the immersion time should be held to a minimum to avoid unnecessary loss of therapeutic agent into the bath.

In some embodiments, the sheet or membrane comprises at least one biodegradable material in a wt % of about 99.5%, 99%, 98%, 97%, 96%, 95%, 94%, 93%, 92%, 91%, 90%, 89%, 88%, 87%, 86%, 85%, 84%, 83%, 82%, 81%, 80%, 79%, 78%, 76%, 75%, 74%, 73%, 72%, 71%, 70%, 65%, 60%, 55%, 50%, 45%, 35%, 25%, 20%, 15%, 10%, or 5% based on the total weight of the sheet or membrane and the remainder is active and/or inactive pharmaceutical ingredients.

In some embodiments, the at least one biodegradable polymer comprises poly(lactic-co-glycolide) (PLGA) or poly(orthoester) (POE) or a combination thereof. The poly(lactic-co-glycolide) may comprise a mixture of polyglycolide (PGA) and polylactide and in some embodiments, in the mixture, there is more polylactide than polyglycolide. In various embodiments there is 100% polylactide and 0% polyglycolide; 95% polylactide and 5% polyglycolide; 90% polylactide and 10% polyglycolide; 85% polylactide and 15% polyglycolide; 80% polylactide and 20% polyglycolide; 75% polylactide and 25% polyglycolide; 70% polylactide and 30% polyglycolide; 65% polylactide and 35% polyglycolide; 60% polylactide and 40% polyglycolide; 55% polylactide and 45% polyglycolide; 50% polylactide and 50% polyglycolide; 45% polylactide and 55% polyglycolide; 40% polylactide and 60% polyglycolide; 35% polylactide and 65% polyglycolide; 30% polylactide and 70% polyglycolide; 25% polylactide and 75% polyglycolide; 20% polylactide and 80% polyglycolide; 15% polylactide and 85% polyglycolide; 10% polylactide and 90% polyglycolide; 5% polylactide and 95% polyglycolide; and 0% polylactide and 100% polyglycolide.

In various embodiments that comprise both polylactide and polyglycolide; there is at least 95% polylactide; at least 90% polylactide; at least 85% polylactide; at least 80% polylactide; at least 75% polylactide; at least 70% polylactide; at least 65% polylactide; at least 60% polylactide; at least 55%; at least 50% polylactide; at least 45% polylactide; at least 40% polylactide; at least 35% polylactide; at least 30% polylactide; at least 25% polylactide; at least 20% polylactide; at least 15% polylactide; at least 10% polylactide; or at least 5% polylactide; and the remainder of the biopolymer is polyglycolide.

In some embodiments, the at least one biodegradable polymer comprises poly(D,L-lactide-co-caprolactone), or poly(L-lactide-co-caprolactone) or copolymers thereof or a combination thereof. The molar ratio of D,L-lactide or L-lactide to caprolactone in the poly(D,L-lactide-co-caprolactone), or poly(L-lactide-co-caprolactone) is 95% D,L-lactide or L-lactide and 5% caprolactone; 90% D,L-lactide or L-lactide and 10% caprolactone; 85% D,L-lactide or L-lactide and 15% caprolactone; 80% D,L-lactide or L-lactide and 20% caprolactone; 75% D,L-lactide or L-lactide and 25% caprolactone; 70% D,L-lactide or L-lactide and 30% caprolactone; 65% D,L-lactide or L-lactide and 35% caprolactone; 60% D,L-lactide or L-lactide and 40% caprolactone; 55% D,L-lactide or L-lactide and 45% caprolactone; 50% D,L-lactide or L-lactide and 50% caprolactone; 45% D,L-lactide or L-lactide and 55% caprolactone; 40% D,L-lactide or L-lactide and 60% caprolactone; 35% D,L-lactide or L-lactide and 65% caprolactone; 30% D,L-lactide or L-lactide and 70% caprolactone; 25% D,L-lactide or L-lactide and 75% caprolactone; 20% D,L-lactide or L-lactide and 80% caprolactone; 15% D,L-lactide or L-lactide and 85% caprolactone; 10% D,L-lactide or L-lactide and 90% caprolactone; or 5% D,L-lactide or L-lactide and 95% caprolactone or copolymers thereof or combinations thereof. In various embodiments, the medical device comprises polymers and copolymers containing various molar ratios of PEG, lactide, glycolide and/or caprolactone.

In some embodiments, at least 75% of the particles (e.g., therapeutic agent, sheet or membrane, adhesive) have a size from about 20 micrometer to about 180 micrometers. In some embodiments, at least 85% of the particles have a size from about 20 micrometers to about 180 micrometers. In some embodiments, at least 95% of the particles (e.g., therapeutic agent, sheet or membrane, adhesive) have a size from about 20 micrometer to about 180 micrometers. In some embodiments, all of the particles have a size from about 20 micrometer to about 180 micrometers.

In some embodiments, there is a sheet or membrane comprising therapeutic agent and a polymer, wherein the polymer is one more of various embodiments, the sheet or membrane comprises poly(lactide-co-glycolide) (PLGA), polylactide (PLA), polyglycolide (PGA), D-lactide, D,L-lactide, L-lactide, D,L-lactide-co-ε-caprolactone, D,L-lactide-co-glycolide-co-ε-caprolactone or a combination thereof.

It will be apparent to those skilled in the art that various modifications and variations can be made to various embodiments described herein without departing from the spirit or scope of the teachings herein. Thus, it is intended that various embodiments cover other modifications and variations of various embodiments within the scope of the present teachings.

What is claimed is:

1. A medical device for delivering an adhesive membrane at or near a target tissue site, the medical device comprising a cannula defining a longitudinal axis and having a proximal end and a distal end, the proximal end of the cannula having an opening configured to receive a flowable material, the distal end of the cannula connected to an expandable member, the expandable member having an interior configured to receive the flowable material and a surface aligned with and contacting at least a portion of the adhesive membrane, the adhesive membrane configured to attach to a select tissue surface of the target tissue site in an open position.

2. A medical device according to claim 1, wherein the expandable member comprises a balloon and the balloon is configured to expand on receiving flowable material and move the adhesive membrane against the select tissue surface to seal the select tissue surface in the open position.

3. A medical device according to claim 2, wherein the balloon comprises a port configured to couple to the cannula.

4. A medical device according to claim 2, wherein the balloon is configured to expand in a direction transverse to the cannula to move the adhesive membrane against the select tissue surface in the open position.

5. A medical device according to claim 2, wherein the balloon is configured to expand more in length than in height.

6. A medical device according to claim 2, wherein the balloon is contiguous with the adhesive material when the balloon is expanded.

7. A medical device according to claim 2, wherein the balloon has a length longer or the same size as the adhesive material when the balloon is expanded.

8. A medical device according to claim 2, wherein the balloon is configured to expand when a liquid or gas is contained within the balloon or the proximal end is configured to receive a plunger slidably movable within the interior of the cannula.

9. A medical device according to claim 2, wherein the adhesive membrane comprises a sheet having an adhesive disposed in or on at least one of its surfaces.

10. A medical device according to claim 9, wherein the adhesive sheet comprises a folding portion and two edges, each edge disposed substantially parallel to each other and above the balloon and each edge contacting the cannula to form a pocket and the balloon disposed within the pocket.

11. A medical device according to claim 9, wherein the adhesive sheet comprises (i) an adhesive disposed in or on the sheet at discrete positions; (ii) wherein the adhesive sheet comprises adhesive disposed on each of its sides; (iii) a surface area that is smaller than a surface area of the balloon; (iv) an expandable material; or (v) a therapeutic agent disposed in or on the adhesive sheet.

12. A medical device according to claim 9, wherein (i) the balloon is attached to the adhesive sheet by an adhesive disposed at discrete positions on the balloon; (ii) the balloon is configured to be separated from the adhesive sheet on application of a pulling force or turning force on the cannula; (iii) the balloon is configured to be separated from the adhesive sheet when in an open position and connected to the adhesive sheet when in a closed position; or (iv) the balloon comprises a lubricant on a surface that contacts the adhesive sheet.

13. A medical device according to claim 9, wherein the select tissue surface is an intervertebral disc.

14. A medical device for delivering an adhesive sheet adjacent to a target tissue site, the medical device comprising a cannula defining a longitudinal axis and having a proximal end and a distal end, the proximal end of the cannula having an opening configured to receive a flowable material, the distal end of the cannula connected to a balloon, the balloon having an interior configured to receive the flowable material and a surface aligned with and contacting at least a portion of the adhesive sheet, the balloon configured to move in an inflated position and an a deflated position, wherein in the inflated position, the adhesive sheet conforms to a select tissue surface of the target tissue site and in the deflated position, the adhesive sheet is in a closed position.

15. A medical device according to claim 14, wherein (i) the balloon is attached to the adhesive sheet by an adhesive disposed at discrete positions on the balloon; (ii) the balloon is configured to be separated from the adhesive sheet on application of a pulling force or turning force to the cannula; or (iii) the balloon comprises a lubricant on a surface that contacts the adhesive sheet.

16. A medical device according to claim 14, wherein the select tissue surface is an intervertebral disc.

17. A method for treating a target tissue site, the method comprising: inserting a balloon adjacent to the target tissue site, the balloon having a surface aligned with and connected to at least a portion of an adhesive sheet, the balloon configured to move from a deflated position when the adhesive sheet is in a closed position to an inflated position when the adhesive sheet is in an open position; positioning the adhesive sheet adjacent to a select tissue surface of the target tissue site; inflating the balloon to move the adhesive sheet to an open position so as to conform the adhesive sheet to the select tissue surface and adhere the adhesive sheet to the select tissue surface of the target tissue site.

18. A method for treating a target tissue site according to claim 17, further comprising deflating the balloon and removing it from the target tissue site.

19. A method for treating a target tissue site according to claim 17, wherein the adhesive sheet has an adhesive disposed on one of its sides.

20. A method for treating a target tissue site according to claim 17, wherein the target tissue site is a nucleus pulposis or annulus fibrosis of an intervertebral disc, a kidney, blood vessels, lungs, heart or liver.
